(19) United States
(12) Patent Application Publication
(10) Pub. No.: US 2024/0352488 A1
(43) Pub. Date: Oct. 24, 2024

(54) GUIDE RNA AND USES THEREOF

(71) Applicant: UCL BUSINESS LTD, London, Greater London (GB)

(72) Inventors: Claire BOOTH, London, Greater London (GB); Benjamin C. HOUGHTON, London, Greater London (GB)

(73) Assignee: UCL BUSINESS LTD, London, Greater London (GB)

(21) Appl. No.: 18/701,115

(22) PCT Filed: Oct. 17, 2022
(86) PCT No.: PCT/EP2022/078863

§ 371 Date: Apr. 12, 2024

(30) Foreign Application Priority Data
Oct. 18, 2021 (GB) 2114871.3

(51) Int. Cl. C12N 15/90; C12N 5/0783; C12N 9/22; C12N 15/11; C12N 15/86

(52) U.S. Cl. CPC C12N 15/907; C12N 5/0636; C12N 9/22; C12N 15/11; C12N 15/86; C12N 2310/20; C12N 2750/14143

(57) ABSTRACT

A guide RNA comprising (17) to (24) nucleotides which are complementary to exon 1 of the human SH2D1A or the complement thereof. Ribonuclear proteins (RNP) complexes comprising the guide RNA and a Cas nuclease, and gene editing kits comprising the ribonuclear protein (RNP) complexes are also disclosed, including the use of these components in therapy, particularly for SAP-mediated diseases.

### BACKGROUND

X linked lymphoproliferative disease (XLP) is a rare inherited immune system disorder, affecting 1-2:1,000,000 births. It arises due to mutations and deletions in the SH2D1A gene, which encodes an intracellular adaptor protein SLAM-associated protein (SAP) that is critical for relaying signals received at the cell surface by SLAM family receptors. SAP is a small 128 amino acid cytoplasmic protein consisting of a single Src homology-2 (SH2) domain and a short C-terminal tail. By binding to specific tyrosine-based motifs in the cytoplasmic tail of SLAM family receptors—such as SLAM, 2B4, NTB-A, Ly9 CD84 and CRACC—via an arginine residue in the SH2 domain, SAP can recruit addition proteins that can activate downstream signalling cascades.

In the absence of SAP, several immune functions are affected, including reduced T cell and NK cell cytotoxicity, a lack of NKT cell development, defective CD4 T follicular helper (T_{FH}) cell help to B cells leading to abnormal humoral function, and a reduced sensitivity to restimulation-induced cell death (RICD) that contributes to unconstrained immune responses to viral infection. These deficits give rise to a range of clinical manifestations, including haemophagocytic lymphohistiocytosis (HLH), dysgammaglobulinaemia, lymphoma and autoimmunity.

Treatment options for XLP patients are currently limited and the only curative therapy is a haematopoietic stem cell transplant (HSCT), however, outcomes can be poor in the mismatched donor setting, as patients are at risk of graft-versus host disease (GvHD) amongst other complications. It has previously been shown that lentiviral gene addition can restore SAP protein expression and immune function when delivered to HSC and T cells, in several in vitro and in vivo models. However, SAP has a tightly controlled expression profile, limited to T_{convs} (not T_{reg}), NK and NKT cells. Within T cell subsets, SAP expression levels are upregulated after TCR engagement and alter with memory or effector phenotypes, indicating an importance of finely tuned control and giving rise to concern that uncontrolled expression of this important signalling molecule in a conventional gene therapy procedure could cause further dysregulation.

There is therefore a need to develop improved tools to regulate the expression of the SAP protein, preferably by developing tools that maintain endogenous expression of the SAP protein, which can be used in the treatment of XLP.

### SUMMARY OF INVENTION

In a first aspect, there is provided a guide RNA comprising 17 to 24 nucleotides which are complementary to exon 1 of human SH2D1A. In some embodiments, the guide RNA comprises one of SEQ ID NO: 5-18. In some embodiments, the guide RNA comprises 20 nucleotides complementary to exon 1 of human SH2D1A. In some embodiments, the guide RNA comprises 21 nucleotides complementary to exon 1 of human SH2D1A.

In a second aspect, there is provided a ribonuclear protein (RNP) complex comprising a Cas enzyme and the guide RNA of the first aspect.

In a third aspect, there is provided construct encoding the guide RNA of the first aspect.

In a fourth aspect, there is provided a gene editing kit comprising
the ribonuclear protein complex of the second aspect, and
a donor sequence or a construct encoding for a donor sequence. In some embodiments, the donor sequence encodes for SAP.

In a fifth aspect, there is provided an in vitro method of forming a double strand break within exon 1 of the human SH2D1A gene, the method comprising contacting the cell with the RNP complex of the second aspect. In some embodiments, the cell is a T cell. In other embodiments the cell is a haematopoietic stem cell.

In a sixth aspect, there is provided an in vitro method of editing the SH2D1A gene, the method comprising contacting a cell with the gene editing kit of the fourth aspect. In some embodiments, the cell is a T cell. In other embodiments the cell is a haematopoietic stem cell.

In a seventh aspect, there is provided the guide RNA of the first aspect, the RNP complex of the second aspect, or the gene editing kit of the fourth aspect for use in therapy.

In an eighth aspect, there is provided the guide RNA of the first aspect, the RNP complex of the second aspect, or the gene editing kit of the fourth aspect for use in the treatment of a SAP-mediated disease.

In a ninth aspect, there is provided the gene editing kit of the fourth aspect for use in the treatment of X-linked lymphoproliferative disease.

Also disclosed herein, is a guide RNA comprising one of SEQ ID NO: 5-18. An RNP complex comprising the guide RNA comprising one of SEQ ID NO: 5-18 and a Cas endonuclease is also disclosed, along with gene editing kits and methods using the guide RNA comprising one of SEQ ID NO: 5-18.

Also disclosed herein is a CRISPR/Cas kit comprising a Cas enzyme and i) the guide RNA of the first aspect or ii) the construct of the third aspect. The kit can be used to form the RNP complex of the second aspect.

The present invention provides an effective way to manipulate SAP expression using a CRISPR/Cas platform. Such a gene editing approach, using site specific nucleases and a homology directed repair template to place a donor sequence (e.g. a corrective SAP cDNA) under the control of the full native promoter, could harness more of the endogenous regulatory elements that govern SAP expression, which may be used to provide an optimal therapy. The present genome editing platform may provide an autologous intervention for patients lacking a suitable haematopoietic stem cell donor for transplant. Here, we demonstrate gene editing of the SH2D1A locus in T cells, which may be used to provide a potentially lifesaving cure of XLP patients. This disclosure also provides preliminary evidence that this approach can be used for editing the SH2D1A locus in haematopoietic stem cells. This would be particularly effective since this approach could correct SAP-dependent functions in all affected immune lineages (including T, NK and NKT cells), for the lifetime of the patient. The present invention also provides a method for effectively altering SH2D1A and SAP expression.

Unexpectedly both CRISPR/Cas9 and CRISPR/Cas12a platforms demonstrated high editing efficiency in T cells. This was observed particularly for the CRISPR/Cas9 platform which showed >90% knockdown of SAP protein, which was comparable, and in some instances better, than the knockdown observed using a comparative TALEN platform. This result is surprising since it is reported that TALEN platforms outperform Cas9 platforms when editing heterochromatin target sites (see Jain et al, Nature Communications, 12, 606 (2021). Kuo et al and Schumann et al, have also reported RNP editing of T cells at a much lower efficiency than what is demonstrated in the present invention, albeit for targeting a different gene; see Cell Reports, 23, 2606-2616 May 2019, https://doi.org/10.1016/j.celrep.2018.04.103) and PNAS, vol. 112, 33, 10437-10442, www.pnas.org/cgi/doi/10.1073/pnas.1512503112 respectively). Preliminary experiments also suggest that this approach is translatable to haematopoietic stem cells.

While CRISPR/Cas12a platform showed a moderate editing activity (˜50% knockdown), this platform importantly demonstrated no off-target effects. Such an approach is therefore improved over a comparative TALEN platform which demonstrated off-target effects against the gene encoding for TET1. Off-target effects against TET1 are considered to be particularly disadvantageous because TET1 dysregulation is connected to malignancy. TET1 is also heavily implicated in epigenetic regulation since it is associated with active demethylation of 5-methylcytosine (5-mC). Extensive evidence suggests that the TET family of enzymes, including TET1, can oxidize 5-mC to 5-hydroxymethylcytosine (5-hmC), 5-formylcytosine (5-fC) and 5-carboxycytosine (5-caC). 5-fC and 5-caC can then be repaired by the base excision repair pathway back to an unmodified C. TET1 is also understood to facilitate the nuclear reprogramming of somatic cells to induced pluripotent stem cells (iPSCs). TALEN platforms may therefore be more cytotoxic than CRISPR/Cas platforms.

Furthermore, since the Cas12a nuclease creates a staggered cut, as opposed to the blunt cut generated by Cas9. Cas12a creates a 5 bp overhang (downstream of PAM, from 18 nucleotides on the non-target strand, to 23 on the target strand) which may more efficiently stimulate homology directed repair (HDR). As a result, the double strand break created by the nucleases can be harnessed to seamlessly insert therapeutic sequences by supplying a HDR template. Indeed, despite having a cutting activity almost half that of the Cas9 RNPs, the Cas12a RNPs were able to mediate similar levels of HDR as Cas9. Both Cas9 and Cas12a showed a high rate of HDR with up to 30% GFP positive cells.

It is also expected that CRISPR delivery by RNP complex is particularly advantageous over other gene-editing approaches, such as TALENs, due to a short-term burst of nuclease expression that can reduce off-target effects as compared to mRNA delivery.

The present invention therefore provides a safe and efficient way of editing the SH2D1A locus, which is improved over other methods of altering SAP expression, which is translatable to different cell types.

### BRIEF DESCRIPTION OF FIGURES

FIG. 1 shows the assessment of on and off target TALEN-, Cas9 and Cas12a-mediated activity. [A] Schematic diagram of the SH2D1A locus showing the start of the exon 1, TALEN-L and -R binding sites, Cas9 and Cas12a gRNA DNA target sequences [B] T cell genome editing experimental timeline [C] % SAP negative healthy control T cells at d6 post nucleofection with TALEN mRNA, Cas9 or Cas12a RNPs (n=3-6, mean, SEM) [D] % INDEL frequency by TIDE analysis of sanger sequencing data of PCR amplicons amplified from nuclease treated T cell genomic DNA (n=3-7, mean, SEM) [E-G] NGS-generated data of modifications at on target (ON) and in silico predicted off target loci (OT1-10) for [E] TALEN [F] Cas12a-1 and [G] Cas9-3 nucleases, (n=1, treated (Tr) vs untreated (UT), OT sites marked *p<0.0001).

FIG. 2 shows a schematic diagram of the genomic loci for TALEN off target site 2 (OT2) which occurs in TET1. The numbers indicate the chromosome 10 coordinates of the left and right exon-intron boundaries flanking the OT2 site.

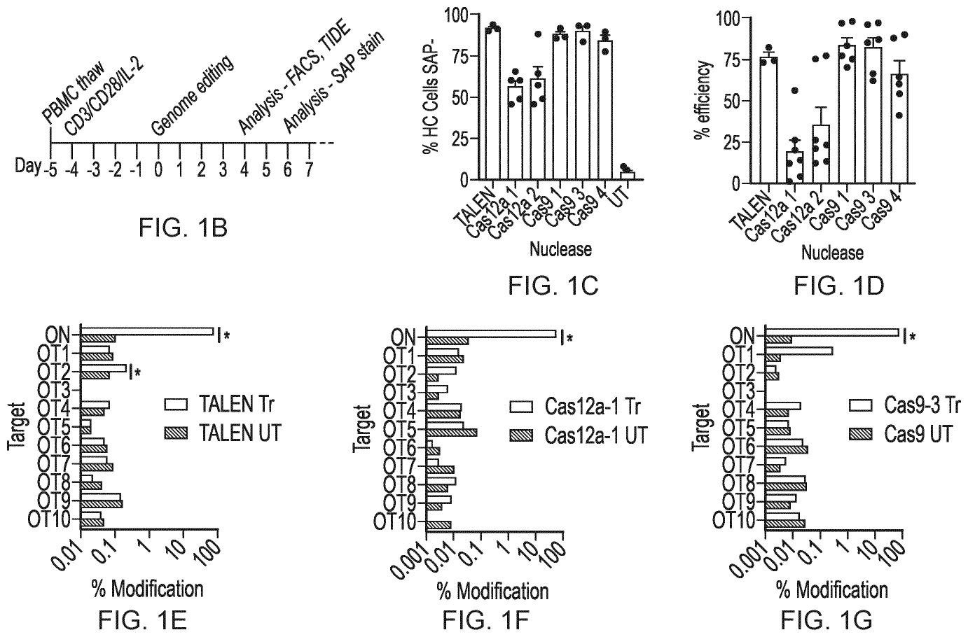

FIG. 3 shows schematic diagrams of HDR donor sequences designed for targeted insertion at the SH2D1A locus: G7bc (Homology arm left (HAL)-bovine growth hormone poly adenylation signal (bGHpA), EF1aShort (EFS), enhanced green fluorescent protein (GFP), woodchuck post-transcriptional regulatory element (WPRE), SV40pA, homology arm right (HAR)); G15 (HAL, coSAP, P2A, GFP, WPRE, SV40pA, HAR); G16 (HAL, coSAP, P2A, GFP, 1764bp proximal 3′untranslated region (SAP_{1764bp}UTR), HAR). All constructs are flanked at the 5 and 3′ by inverted terminal repeats (ITRs) of an AAV2 viral genome (not shown).

FIG. 4 shows the % GFP+ T cells transduced with a range of MOIs (multiplicity of infection) of G7bc AAV in HS or HS-free conditions 15 minutes post-nucleofection with [Left] Cas12a-1 RNP or [Right] Cas9-3 RNP (N=2, mean, SD).

FIG. 5 shows the cutting efficiency of various Cas9/Cas12a RNPs targeting the SH2D1a exon 1 in both T cells and haematopoietic stem cells.

FIG. 6 shows evidence of T-cell editing using CRISPR-Cas nucleases and a SAP-containing homology donors—T cells were transduced with constructs G7bc AAV (control), G15 AAV or G16 AAV within 15 minutes of electroporation with Cas9-3 RNP.

FIG. 7 shows the frequency of INDEL formation in HSCs using Cas9-1, Cas9-3, Cas9-4 and TALEN.

### DETAILED DESCRIPTION

The details, examples and preferences provided in relation to one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

The terms “treatment” and “treating” herein refer to an approach for obtaining beneficial or desired results in a patient, which includes a prophylactic benefit and a therapeutic benefit.

“Therapeutic benefit” refers to eradication, amelioration or slowing the progression of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

“Prophylactic benefit” refers to delaying or eliminating the appearance of a disease or condition, delaying or eliminating the onset of symptoms of a disease or condition, slowing, halting, or reversing the progression of a disease or condition, or any combination thereof. In the context of the present invention, the prophylactic benefit or effect may involve the prevention of the condition or disease. The guide RNA, RNP complex or gene delivery kit may be administered to a patient at risk of developing a particular disease (e.g. X-linked lymphoproliferative disease), or to a subject reporting one or more of the physiological symptoms of a disease, even though a diagnosis of this disease may not have been made.

The term “effective amount” or “therapeutically effective amount” refers to the amount of the guide RNA, RNP complex or gene delivery kit needed to bring about an acceptable outcome of the therapy as determined by reducing the likelihood of disease as measurable by clinical, biochemical or other indicators that are familiar to those trained in the art. The therapeutically effective amount may vary depending upon the condition, the severity of the condition, the subject, e.g., the weight and age of the subject and the mode of administration and the like, which can readily be determined by one of ordinary skill in the art.

The term “patient” described herein refers to any human subject.

The term “comprising” (and related terms such as “comprise” or “comprises” or “having” or “including”) includes those embodiments, for example, an embodiment of any composition of matter, composition, method, or process, or the like, that “consist of” or “consist essentially of” the described features. The term “comprises” or “comprising” can be used interchangeably with “includes”.

Any genomic or chromosomal position described herein refers to the position on the human genome and associated transcriptome (GRCh38).

Construct defined herein refers to any gene construct that can be used to deliver the donor sequence (e.g. SAP cDNA) into a cell.

When ranges are used herein, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range may vary. Typical experimental variabilities may stem from, for example, changes and adjustments necessary during scale-up from laboratory experimental and manufacturing settings to large scale.

For any sequence described herein, the complementary sequence or reverse complement is also considered part of the disclosure.

The features of any dependent claim may be readily combined with the features of any of the independent claims or other dependent claims, unless context clearly dictates otherwise.

It must be noted that as used herein and in the appended claims, the singular forms “a”, “an”, and “the” include plural referents unless the context clearly dictates otherwise.

Unless defined otherwise, all technical and scientific terms used herein have the same meanings as commonly understood by one of ordinary skill in the art to which this invention belongs. Abbreviations used herein have their conventional meaning within the chemical and biological arts, unless otherwise indicated.

### Guide RNA

In a first aspect there is provided a guide RNA comprising 17 nucleotides to 24 nucleotides which are complementary to exon 1 of human SH2D1A (i.e. the sense strand of exon 1 of human SH2D1A) or its complement thereof (i.e., the anti-sense strand of exon 1 of human SH2D1A). In some embodiments, the guide RNA is complementary to the coding region of exon 1 of human SH2D1A having sequence SEQ ID NO:1. In some embodiments, the guide RNA is complementary to the complement of the coding region of exon 1 of human SH2D1A having sequence SEQ ID NO:2.

Complementary described herein generally refers to 100% sequence complementarity. Sequence complementarity disclosed herein refers to Watson-Crick base pairing in nucleic acids, e.g., wherein A binds with U or T (or modified variants thereof), and wherein C binds with G (or modified variants thereof). Strands of complementary sequence are referred to as sense and anti-sense, with the sense strand being the pre-mRNA that was generated after transcription, with the anti-sense sequence being complementary to the sense sequence.

SH2D1A is otherwise known as SH2 Domain Containing 1A. The coding region of Exon 1 of human SHD21A is located at Chrx: 124346643-124346779. The full exon 1 is located at Chrx 124346563-124346779.

Guide RNA as defined herein refers to one part of a CRISPR/Cas genome editing system, the other part being a CRISPR associated endonuclease (Cas protein). The guide RNA described herein is therefore suitable for use in a CRISPR/Cas genome editing system, and is capable of complexing with a Cas nuclease. The term guide RNA is therefore intended to exclude any naturally transcribed mRNA. The guide RNA in combination with a Cas nuclease, is capable of forming a double strand break (DSB) in exon 1 of human SH2D1A. Guide RNA comprises the RNA equivalent of a target sequence in the human genome, said target sequence having a protospacer adjacent motif (PAM) which is downstream (e.g., for Cas9) or upstream (e.g. for Cas12a) of the target sequence targeted for cleavage by the CRISPR/Cas system, typically named (crRNA). The 17 nucleotide to 24 nucleotide portion of the guide RNA described herein may therefore otherwise be referred to as the crRNA. The guide RNA is capable of forming a complex with the opposite DNA strand from the target sequence. In some embodiments, the guide RNA further comprises a tracrRNA, which together with the 17 nucleotide to 24 nucleotide portion of the guide RNA described herein, is capable of complexing with a Cas nuclease.

In some embodiments, the DSB is formed within 50 bp downstream of the start of the coding region of exon 1 of human SH2D1A, or within 25 bp, or within 10 bp, or within 5 bp of the start of the coding region of exon 1 of human SH2D1A, or directly at the start of exon 1 of the coding region of human SH2D1A (i.e. defined from the start of SEQ NO. 1 or Chrx: 124346643). In some embodiments, the DSB forms blunt ends (e.g. with spCas9). In alternative embodiments, the DSB forms staggered ends (e.g., with Cas12a).

In some embodiments, the DSB is formed within 65 bp downstream of the start of the coding region of exon 1 of human SH2D1A, or within 60 bp, or within 55 bp, or within 45 bp of the start of the coding region of exon 1 of human SH2D1A.

The inventors have observed that the DSB being made closer to start of the coding region of SH2D1A is preferential for HDR-based insertion of therapeutic SAP cDNA sequences, as this allows coverage of a greater number of XLP-causing mutations.

In some embodiments, the guide RNA is capable of binding a target sequence in exon 1 of human SH2D1A (SEQ ID NO:1) or the complement thereof (SEQ ID NO:2), wherein there is a PAM site downstream of the target sequence (i.e. the opposing strand to where the guide RNA binds). In some embodiments, said PAM site is 5′-NGG-3′ where N is G, A, T or C. In some examples, said PAM side is 5′-GGG-3′ or 5′-TGG-3′. This guide RNA would be suitable for use with a spCas9 nuclease.

In some embodiments, the guide RNA is capable of binding to exon 1 of human SH2D1A (SEQ ID NO:1) or the complement thereof (SEQ ID NO:2) wherein there is a PAM site upstream of the target sequence (i.e. the opposing strand to where the guide RNA binds). In some embodiments, said PAM site is 5′-TTTV-3′ where V is G, A, C. In some examples, said PAM side is 5′-TTTG-3′ or 5′-TTTC-3′. This guide RNA would be suitable for use with a Cas12a nuclease, e.g., asCas12a nuclease.

In some embodiments, the guide RNA comprises 17 nucleotides which are complementary to exon 1 of human SH2D1A (SEQ ID NO: 1) or the complement thereof (SEQ ID NO:2). In some embodiments, the guide RNA comprises 18 nucleotides, or 19 nucleotides, or 20 nucleotides, or 21 nucleotides, or 22 nucleotides, or 23 nucleotides, or 24 nucleotides. In a preferred embodiment, the guide RNA comprises 20 nucleotides or 21 nucleotides which are complementary to exon 1 of human SH2D1A (SEQ ID NO: 1) or the complement thereof (SEQ ID NO:2). In some embodiments, the guide RNA comprises 17 to 24 nucleotides that are complementary to SEQ ID NO: 3 or SEQ ID NO: 4, i.e., which corresponds to the first portion of exon 1 of human SH2D1A.

In some embodiments, the guide RNA further comprises a scaffold sequence for a Cas nuclease. In an embodiment, the scaffold sequence is a scaffold sequence for a Cas9 nuclease, for example, spCas9 nuclease.

In some embodiments, the guide RNA further comprises a tracrRNA.

In some embodiments, the guide is a single guide RNA (sgRNA). In other words, the guide RNA is formed of only one RNA molecule. In some embodiments, the single guide RNA comprises a tracrRNA and the RNA comprising the 17 nucleotides to 24 nucleotides complementary to the first coding exon of human SH2D1A (SEQ ID NO:1) or the complement thereof (SEQ ID NO:2), and wherein the tracrRNA and the RNA comprising the 17 nucleotides to 24 nucleotides are covalently linked.

In some embodiments, the single guide RNA is synthetic, i.e., formed using oligonucleotide synthesizers. In some embodiments, the single guide RNA is a chemically modified single guide RNA, in other words, at least one ribonucleonotide is modified. In some embodiments, the single guide RNA may comprise a 5′-OH or 3-OH end blocking group. In some embodiments, the single guide RNA may comprise one or more of 2′O-methyl RNA bases, phosphorothioated RNA bases and/or a 5′ or 2′ end-blocking modification.

In some embodiments the single guide RNA is absent of tracrRNA (e.g., suitable for use with a Cas12a nuclease.

In alternative embodiments, the guide RNA is a two part molecule, one part comprising a tracrRNA and the other comprising the crRNA (i.e. 17 nucleotides to 24 nucleotides which is complementary to the first coding exon of human SH2D1A (SEQ ID NO:1) or the complement thereof (SEQ ID NO:2)). In some embodiments, the first part is ligated to or complexed to the second part. Such systems may be used with a Cas9, or any Cas9 nuclease referred to herein.

In some embodiments, the guide RNA comprises one of SEQ ID NO. 5-12. These guide RNA sequences are suitable for guiding a spCas9 nuclease and are demonstrate to have excellent cutting efficiency in certain cells, e.g., T cells.

In some embodiments, the guide RNA comprises one of SEQ ID NO. 5-6. SEQ ID NO:5 corresponds to the Cas9-1 sequence in the application examples, and SEQ ID NO:6 is a 17 nucleotide truncated variant thereof (Cas9-1ₜ).

In some embodiments, the guide RNA comprises one of SEQ ID NO. 7-8. SEQ ID NO:7 corresponds to the Cas9-2 sequence in the application examples, and SEQ ID NO:8 is a 17 nucleotide truncated variant thereof (Cas9-2ₜ).

In some embodiments, the guide RNA comprises one of SEQ ID NO. 9-10. SEQ ID NO:9 corresponds to the Cas9-3 sequence which is used in the application examples, and SEQ ID NO: 10 is a 17 nucleotide truncated variant thereof (Cas3-2ₜ).

In some embodiments, the guide RNA comprises one of SEQ ID NO. 11-12. SEQ ID NO:11 corresponds to the Cas4-3 sequence which is used in the application examples, SEQ ID NO:12 is a 17 nucleotide truncated variant thereof (Cas4-2ₜ).

In some examples, the guide RNA comprises one of SEQ ID NO 5-6, SEQ ID NO 9-10, or SEQ ID NO 11-12. These guide RNA sequences enable good cutting efficiency with spCas9 in both T cells and HSCs. In some examples, the guide RNA comprises SEQ ID NO. 9-10. These guide RNAs may be preferred since they are shown to have good editing efficiency while also cutting towards the start of the first coding exon of human SH2D1A. In some examples, the guide RNA comprises SEQ ID NO: 9.

In some embodiments, the guide RNA comprises one of SEQ ID NO. 13-18. These guide RNA sequences are suitable for guiding a Cas12a nuclease and are demonstrate to have excellent cutting efficiency in certain cells, e.g., T cells.

In some embodiments, the guide RNA comprises one of SEQ ID NO. 13-14. SEQ ID NO. 13 corresponds to the Cas12a-1 sequence used in the application examples, and SEQ ID NO. 14 is a 17 nucleotide truncated variant thereof (Cas12a-1ₜ). In some examples, these guide RNAs are preferred since they are shown to have the best editing efficiency for Cas12a in certain examples (e.g. in HSCs) while also cutting towards the start of the first coding exon of human SH2D1A. In some examples, the guide RNA comprises SEQ ID NO: 13.

In some embodiments, the guide RNA comprises one of SEQ ID NO. 15-16. SEQ ID NO. 15 corresponds to the Cas12a-2 sequence in the application examples, SEQ ID NO: 16 is a 17 nucleotide truncated variant thereof (Cas12a-2ₜ).

In some embodiments, the guide RNA comprises one of SEQ ID NO. 17-18. SEQ ID NO. 17 corresponds to the Cas12a-3 sequence in the application examples, SEQ ID NO: 18 is a 17 nucleotide truncated variant thereof (Cas12a-3ₜ).

### Ribonuclear Protein Complex

In a second aspect, there is provided a ribonuclear protein (RNP) complex comprising a Cas enzyme and the guide RNA of the first aspect. Cas enzymes may be used interchangeably with the terms Cas nuclease or Cas endonucelease or Cas protein.

In some embodiments, the Cas enzyme is a Cas9 enzyme. In some embodiments the Cas9 enzyme is a spCas9 enzyme. In some embodiments, the spCas9 enzyme is a mutant spCas9 enzyme, such as S.p. HiFi Cas9 Nuclease V3 (sold by IDT), SpCas9-HF1, HypaCas9, eCas9-1.1. In some embodiments, the Cas9 enzyme is a spCas9 enzyme and the guide RNA comprises one of SEQ ID NO 5-12. In some embodiments, the molar ratio of spCas9 enzyme to guide RNA is about 1:0.5 to about 1:30, or from 1:0.5 to about 1:25, or from 1:0.5 to about 1:10, or from 1:0.5 to about 1:5, or from about 1:0.5 to about 1:2.5, more preferably from 1:1 to about 1:5, optionally about 1:2.5. In some embodiments, the molar ratio of spCas9 enzyme to guide RNA is less than or equal to 1:0.5, or less than 1:1, or less than 1:3, or less than 1:4, or less than 1:5, or less than or equal to 1:6, or less than 1:7, or less than 1:8, or less than 1:9. In some embodiments, the molar ratio of spCas9 enzyme to guide RNA is greater than about or equal to 1:10, or greater than about 1:9, or greater than about 1:8, or greater than about 1:7, or greater than about 1:6, or greater than or equal to about 1:5, or greater than equal to 1:4, or greater than equal to 1:3, or greater than equal to 1:2, or greater than equal to 1:1.

In some embodiments, the Cas enzyme is a Cas12a enzyme. In some embodiments, the Cas12a enzyme is an asCas12a enzyme. In some embodiments, the Cas12 enzyme is a mutant Cas12a enzyme, for example, a mutant asCas12a enzyme, such as asCas12a (Cpf1) Ultra, sold by IDT), lbCas12a and engCas12a. In some embodiments, the Cas12a enzyme is asCas12a enzyme and the guide RNA comprises one of SEQ ID NO 13-18. In some embodiments, the molar ratio of Cas12a enzyme to guide RNA is less than or equal to 1:0.5, or less than 1:1, or less than 1:3, or less than 1:4, or less than 1:5, or less than or equal to 1:6, or less than 1:7, or less than 1:8, or less than 1:9. In some embodiments, the molar ratio of Cas12a enzyme to guide RNA is greater than about or equal to 1:10, or greater than about 1:9, or greater than about 1:8, or greater than about 1:7, or greater than about 1:6, or greater than or equal to about 1:5, or greater than equal to 1:4, or greater than equal to 1:3, or greater than equal to 1:2, or greater than equal to 1:1.

In preferred embodiments, the RNP complex is formed before entering the cell. In other embodiments, the RNP complex is formed within the cell.

In a third aspect, there is provided construct encoding the guide RNA of the first aspect. The construct may be any suitable construct, e.g., a viral vector, such as an AAV vector.

In a fourth aspect, there is provided a gene editing kit comprising the ribonuclear protein complex of the second aspect, and a donor sequence or a construct encoding for a donor sequence (i.e., a HDR donor sequence). In some embodiments, the donor sequence comprises a sequence that encodes for SAP. In some embodiments, the construct encodes for a sequence that encodes for SAP. In some embodiments, the construct encodes for codon optimised SAP cDNA (coSAP), e.g., wherein the construct comprises SEQ ID NO: 19. In some embodiments, the construct encodes for GFP. e.g., wherein the construct comprises SEQ ID NO: 20. Typically, the construct comprises homologous sequences at either end which are complementary to the sequences on either side of the cleavage site. The homologous sequences may range from 100 to 1000 bp, or from about 700 to 1000 bp, usually about 850 bp of the regions that are complementary to the sequences on either side of the cleavage site. This may cover at least a portion of the 5′-UTR and in some examples at least a portion of the promoter region. In some examples the homology arm left (HAL) comprises at least 100 bp in sequence identity with SEQ ID NO: 21, more preferably about 850 bp in sequence identity with SEQ ID NO: 21. In some examples the homology arm right (HAL) comprises at least 100 bp in sequence identity to SEQ ID NO: 22, more preferably about 850 bp in sequence identity to SEQ ID NO: 22. In some embodiments, the construct further comprises a polyadenylation signal, e.g., comprising one or more of SV40pA (corresponding to SEQ ID NO: 23) or bGHpA (SEQ ID NO: 24). In some embodiments, the construct comprises a sequence corresponding to the SH2D1A 3′untranslated region (i.e. corresponding to SEQ ID NO: 25). In alternative embodiments, the construct comprises a sequence corresponding to the woodchuck post-transcriptional element (WPRE), e.g. WPREₘᵤₜ₆, i.e., corresponding to SEQ ID NO: 26. Typically, in some examples, the constructs may be flanked at the 5′ and 3′ by inverted terminal repeats (ITRs), e.g., of an AAV2 viral genome.

In some examples, the construct comprises one of SEQ ID NO: 27-29. SEQ ID NO: 27 is an example construct which encodes for a GFP-only donor G7bc. SEQ ID NO: 28 and 29 are example constructs which coexpress SAP and GFP.

In some embodiments, the gene editing kit comprises a construct encoding for a donor sequence. In some embodiments, the construct is a viral vector. In some embodiments, the construct is an AAV construct. In some embodiments the AAV construct is of serotype AAV6.

In a fifth aspect, there is provided an in vitro method of forming a double strand break within the first coding exon of the human SH2D1A gene, the method comprising contacting the cell with the RNP complex of the second aspect. In some embodiments the cell is a T cell or an HSC. In some embodiments, contacting of the cell with the RNP complex is by electroporation. Also disclosed herein is an in vitro method of forming a double strand break within the first coding exon of the human SH2D1A gene, the method comprising contacting the cell with the guide RNA of the first aspect.

In a sixth aspect, there is provided an in vitro method of editing the SH2D1A gene, the method comprising contacting a cell with the gene editing kit of the fourth aspect. In some embodiments the cell is a T cell or an HSC. In some embodiments, the method comprises contacting the cell with the gene editing kit using electroporation. In some embodiments, the method comprising contacting the cell with the gene editing kit in the absence of serum, e.g., human serum. In other embodiments, the method comprises contacting the cell with the gene editing kit in the presence of one or more of human serum albumin, a small molecule, or polyvinylalcohol. Also disclosed herein is an in vitro method of editing the SH2D1A gene, the method comprising contacting a cell with a) the guide RNA of the first aspect and/or the construct of the third aspect and b) a donor sequence, or a construct encoding for a donor sequence.

In a seventh aspect, there is provided the guide RNA of the first aspect, the RNP complex of the second aspect, or the gene editing kit of the fourth aspect for use in therapy. Also disclosed herein is a pharmaceutical composition comprising the guide RNA of the first aspect, the RNP complex of the second aspect or the gene editing kit of the fourth aspect. The pharmaceutical composition may further comprise one or more excipients or diluents.

In an eighth aspect, there is provided the guide RNA of the first aspect, the RNP complex of the second aspect, or the gene editing kit of the fourth aspect for use in the treatment of a SAP-mediated disease. In some embodiments, the SAP-mediated disease is characterised by overexpression or SAP. In other embodiments, the SAP-mediated disease is characterised by under expression of SAP.

Also disclosed herein, is a method of regulating SAP expression in a patient, the method comprising delivering a therapeutically effective amount of the guide RNA of the first aspect, the RNP complex of the second aspect, or the gene editing kit of the fourth aspect to a cell in a patient for use in the treatment of a SAP-mediated disease. In some embodiments the cell is a T cell or an HSC. In some embodiments, the SAP-mediated disease is X-linked lymphoproliferative disease.

In a ninth aspect, there is provided the gene editing kit of the fourth aspect for use in the treatment, or a method of treatment, of X-linked lymphoproliferative disease. In some embodiments, the method comprises contacting the cell of the patient with the gene-editing kit to alter the expression of SAP. In some embodiments, the cell is a T cell or an HSC. In some embodiments, the method is used for a patient that has no suitable donor for HSCT.

Also disclosed herein, is a method of treatment of X-linked lymphoproliferative disease in a patient, the method comprising delivering a therapeutically effective amount of the gene editing kit of the fourth aspect to a cell in a patient to alter the expression of SAP. In some embodiments the cell is a T cell or an HSC.

### EXAMPLES

### Results

### Targeting the SH2D1A Locus at High Efficiency in T Cells by CRISPR-Cas9 and CRISPR-Cas12a

To determine the feasibility of a gene correction strategy for XLP, we firstly aimed to determine the optimal nuclease platform for creating the site-specific DNA double strand break. Four Cas9 guide RNAs (gRNA) and two Cas12a crRNAs were identified targeting loci early in exon 1 of the SH2D1A gene. This was compared with a comparative TALEN pair. Stimulated PBMCs were nucleofected with either in vitro-transcribed TALEN mRNA, or Cas9- or Cas12a-guide RNA ribonuclear protein (RNP) complexes.

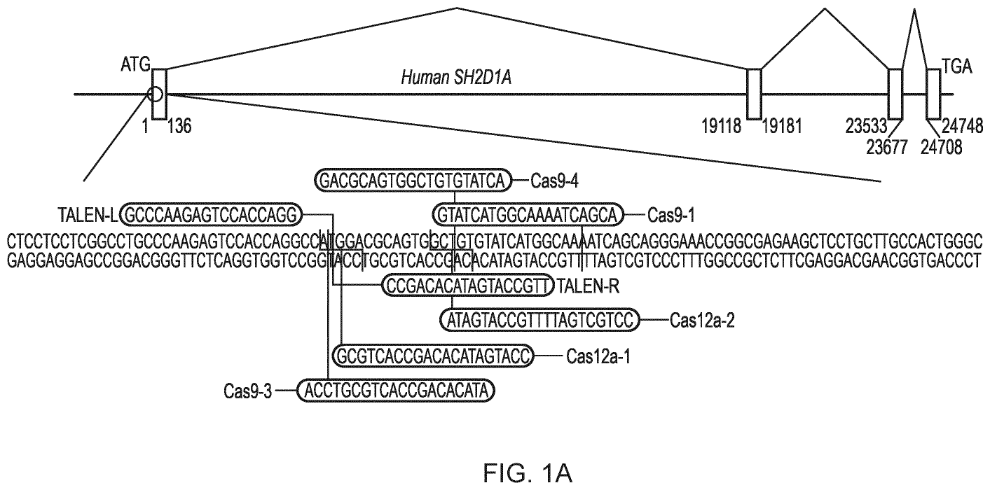

Both Cas9 and Cas12a nuclease platforms mediated efficient gene editing, with Cas9 gRNAs nucleofection resulting in >90% knock down of SAP protein, and Cas12a guides >50% (FIG. 1A). This data was supported by TIDE analysis detection of insertions and deletions (INDELs) created by non-homologous end-joining DNA repair of DNA double strand breaks (FIG. 1D. Cas12a-1 and Cas9-3 were selected for further testing due their proximity to the start of the gene. Surprisingly, the Cas9 platform was at least as efficient, if not more efficient as the TALEN platform.

### Minimal Detection of Off Target Nuclease Activity Across Nuclease Platforms

To assess on and off target cutting at more depth, we performed targeted next generation sequencing (NGS) at the on target (ON) locus and the top predicted off targets (OT1-10) sites for TALENs, Cas12a-1 and Cas9-3 (Supplementary table). NGS confirmed high efficiency modification at the on-target locus across all platforms giving a modification rate of 74%, 75% and 57% for TALEN, Cas9-3 and Cas12a-1 respectively. We detected low frequency off-target activity at two intronic loci (TALEN OT2 and Cas9 OT1) at low frequency (0.22% and 0.29% respectively). TALEN OT2 is in the third intron of the TET1 (Ten-eleven translocation methylcytosine dioxygenase 1) gene while Cas9 OT1 occurs in the twenty fourth intron of RPTOR (Regulatory-associated protein of mTOR). Notably, Cas12a-1 showed no evidence of off-target activity.

### SH2D1A-Targeted DNA Breaks can be Harnessed for HDR-Driven Insertion of DNA

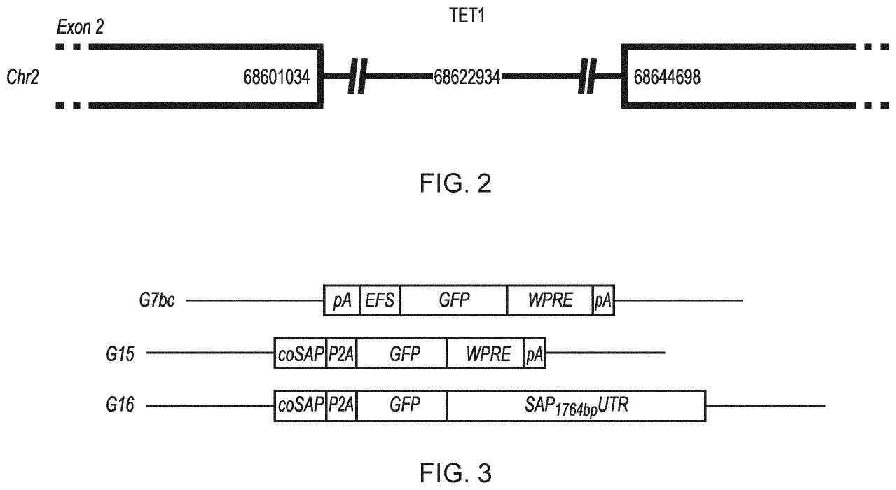

To determine if we could harness the homology directed repair (HDR) pathway to insert a corrective SAP cDNA under its native promoter, we designed a series of donor templates for delivery via AAV6 vector (see FIG. 3). Donors were constructed into an AAV genome using Gibson assembly, to maintain the transition from promoter regions into a codon optimised SAP cDNA without restriction enzyme sequences. A GFP only gene donor was also designed (G7bc). This can be used to shown evidence of HDR-driven insertion. Further, this donor can also be used to create a functional knockout of SAP in healthy control cells thereby providing an additional in vitro model. Other donors were designed for expressing SAP. GFPG15 and G16 donors included a GFP-reporter gene co-expressed with SAP and cleaved during translation via a P2A self-cleaving peptide. HDR efficiency can be assessed by measuring the levels of GFP and SAP protein expression at day 5 post procedure.

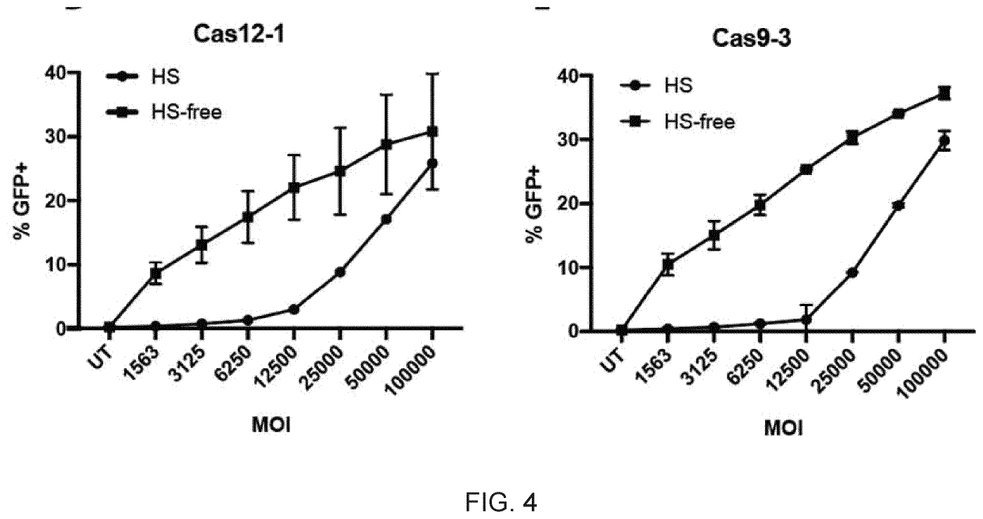

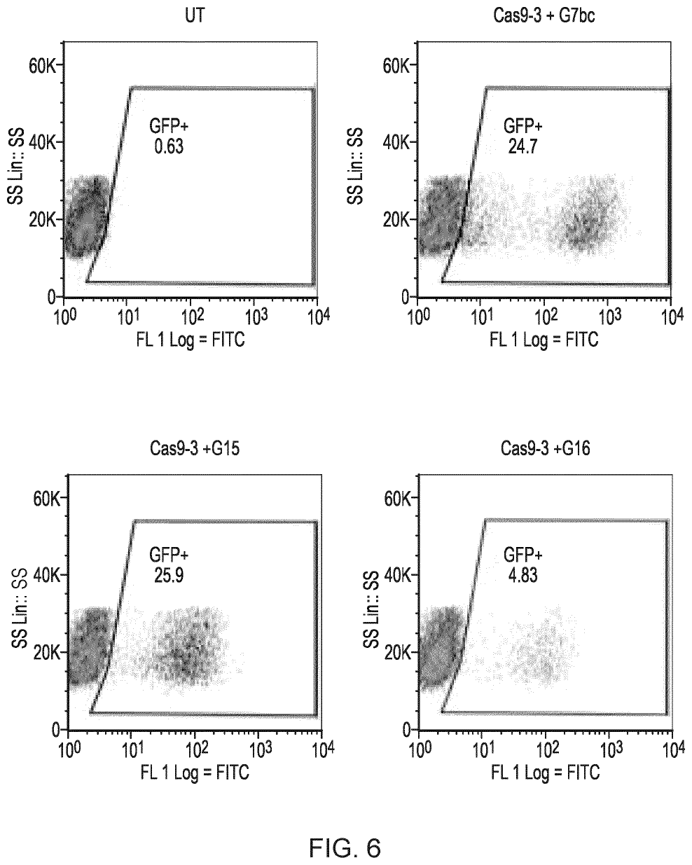

To investigate the gene editing procedure we nucleofected T cells with Cas9-3 or Cas12a-1 nucleases prior to transduction with AAV at a range of MOIs within 15 minutes, in T cell culture media with or without 5% human serum (HA). Those in low HS media were supplemented to full serum culture at 4 hours. For both Cas9-3 and Cas12a-1 platforms we found that cells transduced in the absence of HS had improved rates of HDR over a range of AAV6 MOI (FIG. 4). Furthermore, we observed that Cas9 and Cas12a showed a high rate of HDR with approximately 30% GFP positive cells. GFP data in T cells edited with Cas9-3 RNP and SAP-containing AAV constructs (e.g. G15 and G16) is also shown in FIG. 6.

### INDEL Formation is Increased in HSCs by Using Cas9-1, Cas9-3 or Cas9-4 Nucleases

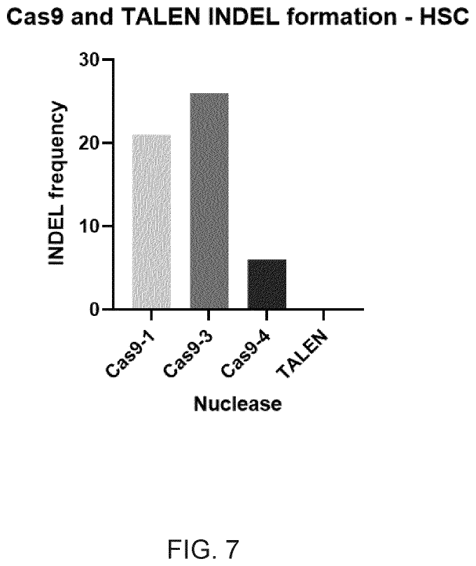

To investigate the efficiency of forming INDEL in HSCs, Cas9-1, Cas9-3 and Cas9-4 were compared against TALEN. The results showed the surprising observation of all three Cas9 nucleases showing increased frequency of INDEL formation compared to TALEN, as shown in FIG. 7.

### Discussion

Haematopoietic stem cell therapy is widely used to treat primary immunodeficiencies, including XLP. However, GvHD remains a significant risk in the mismatched donor setting, leaving an unmet need that could be fulfilled by an autologous gene correction approach. It has previously been shown that lentiviral vectors can be used to deliver a corrective copy of SAP cDNA into HSCs and T cells, to restore immune function in vitro and in vivo models of XLP. However, SAP has a tightly restricted profile that is challenging to replicate using this technology, which may be of particular importance when developing an HSC gene therapy approach. In this study, we show it is plausible to use gene editing technology to create a site-specific insertion of SAP cDNA, hypothesising that this would harness more of the endogenous DNA regulatory mechanisms that govern SAP expression, to provide more physiological expression pattern and therefore a more optimal therapy in both the HSC and T cell setting.

Genome editing is centred on the creation of a site-specific DNA break. We opted to test Cas nuclease technologies to see if they could optimally edit the SH2D1A locus. CRISPR/Cas platforms are preferred to TALEN platforms since it is expected that CRISPR/Cas platforms will be more translatable to different cell types. For example, TALENs may be less efficient in stem cells, in at least because SAP is not expressed in stem cells. Further, CRISPR/Cas platforms using a RNP complex may be safer for use in therapy due to short-term nuclease expression, while also having less off-target effects. Surprisingly, it was found that Cas9 and Cas12a platforms were capable of creating double strand DNA breaks at the SH2D1A locus at high efficiency, with Cas9/CRISPR being comparable in efficiency, and in some instances better, than a comparative TALEN platform. This is unexpected since it has been reported that TALEN platforms outperform Cas9 platforms when editing heterochromatin target sites (see Jain et al, Nature Communications, 12, 606 (2021). Kuo et al and Schumann et al, have also reported RNP editing of T cells at a much lower efficiency than what is demonstrated in the present invention, albeit for a different gene; see Cell Reports, 23, 2606-2616 May 2019, https://doi.org/10.1016/j.celrep.2018.04.103) and PNAS, vol. 112, 33, 10437-10442, www.pnas.org/cgi/doi/10.1073/pnas.1512503112 respectively). Certain Cas9 and Cas12a guide RNAs were found to be particularly effective, either due to their editing efficiency, or due to their translatability into different cell types.

While Cas9 was shown to have a more efficient cutting activity it creates a blunt cut, while Cas12a creates a staggered cut that may more efficiently stimulate HDR. Indeed, despite a cutting activity almost half that of the Cas9 RNPs, our Cas12a RNPs were able to mediate similar levels of HDR as Cas9. As a result, both CRISPR/Cas12a and CRISPR/Cas9 can be used to effectively regulate SAP expression.

We used targeted NGS to investigate on and off target nuclease activity at sites predicted by in silico prediction software. On target amplicons confirmed the highly efficiency genome modification for Cas9 and Cas12a. Modifications at off target loci were absent for Cas12a but were shown for TALEN OT2 and Cas9 OT1. Notably, TALEN OT2 showed an off-target binding to (TET1). Since TET1 is important for haematopoiesis and T cell differentiation and function, and is also important for epigenetic regulation by catalysing the conversion of 5-methylcytosine to 5-hydroxymethylcytosine in DNA, this represents a further disadvantage for reason why the TALEN nuclease platform would not be suitable in therapy.

To determine if we could harness the homology directed repair (HDR) pathway to plausibly insert a corrective SAP cDNA under its native promoter, we designed a series of donor templates for delivery via AAV6 vector. Cas9 and Cas12a showed a good efficiency of HDR, showing approximately 30% GFP positive cells. It was also found that cells transduced in the absence of HS had improved rates of HDR over a range of AAV6 MOI.

We were motivated to optimise the transduction protocols by the high cost associated with performing gene editing at clinical scale. We found that transducing T cells in media without HS leads to significantly improved gene editing at reduced viral MOI, particularly when transduction is performed prior to nucleofection. This approach could offer significant savings in viral production costs needed for each clinical product.

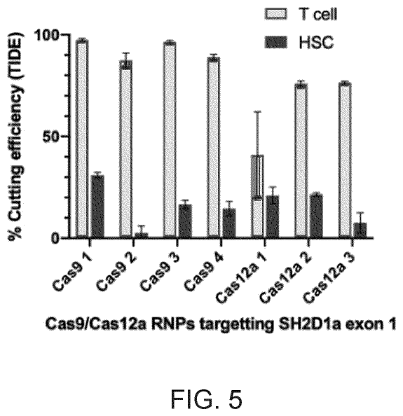

Preliminary studies demonstrate that the CRISPR/Cas9 and CRISPR/Cas12a platforms were also capable of creating double strand DNA breaks in HSCs at the SH2D1A locus with reasonable efficiency (see FIG. 5), with certain guide RNAs performing better than others. This shows that this technology is suitable for and is translatable to an HSC therapy, particularly for patients with XLP lacking a suitable donor for HSCT.

### Materials and Methods

### Cell Culture

Jurkat T cells and lymphoblastoid cell lines were cultured in RPMI containing 10% Foetal bovine serum (FBS) and 1% Penicillin-streptomycin (pen-strep) and passaged twice weekly using 1:10 dilution. HEK293Ts were maintained in DMEM supplemented with 10% FBS and 1% pen-strep and passaged twice weekly. Cells were washed with PBS and released from the culture flask with Trypsin-EDTA (all reagents ThermoFisher Scientific) before collection and neutralisation in complete DMEM and seeding back into culture flask at a 1:10 dilution.

### T Cell Stimulation and Culture

Human peripheral blood mononuclear cells (PBMC) were harvested from whole blood using Ficoll-Paque density centrifugation (GE Healthcare). PBMC were cultured in TexMACS™ Medium (Miltneyi) supplemented with 5% human serum (Sigma) and 1% pen-strep. PBMC were stimulated with Human T-Activator CD3/CD28 Dynabeads (Gibco) at 1:1 ratio, in the presence of 100 U/ml IL-2 (Proleukin) in G-Rex® 24 plates (Wilson Wolf).

TALEN mRNA Synthesis

TALEN pairs were identified and constructed. Plasmid constructs were linearised at the 3′ of the expression cassette using restriction enzyme digest, then purified (Qiagen). mRNA was produced using the T7 mScript™ Standard mRNA Production System according to manufacturer's protocol. Briefly, linearised DNA template was transcribed, treated with DNAse1 and purified (RNeasy Mini-kit, Qiagen), before further reactions for addition of a polyA tail using the supplied enzymes, and Cap 1 structure capping. After a further purification, mRNA integrity was assessed using TapeStation (Agilent) and quantified on NanoDrop Microvolume Spectrophotometer. Left and right TALEN arms were combined at 1:1 ratio, aliquoted (7.5 μg each arm) and stored at −20° C.

### CRISPR-Cas9 and CRISPR-Cas12a Design and RNP Assembly

Potential Cas9 and Cas12a target sites were identified using Benchling online software (www.benchling.com). Cas9 (Alt-R® S.p. HiFi Cas9 Nuclease V3, IDT) and Cas12a (Alt-R® A.s. Cas12a (Cpf1) Ultra, IDT) proteins were complexed to their respective RNA single guides (Cas9 synthetic gRNA, Merck; Cas12a crRNA, IDT), which are chemically modified, at a protein:guide molar ratio of 1:2.5 and 1:2 respectively, at room temperature for 10 minutes immediately prior to nucleofection.

### HSC Culture

CD34+ HSCs were purified from a mobilised (G-CSF/Plerixafor) apheresis taken from consenting healthy donors using CD34 microbeads (Miltenyi) and stored. Thawed cells were washed and plated at 1 million per ml in HSC media supplemented with penicillin/streptomycin and cytokines at 100-300 ng/ml (TPO, FLt3 and SCF). After 2 days of culture, HSC were collected, washed in PBS and counted, between 200,000-1,200,000 cells were electroplated per condition using the Lonza 4D nucelofector. Cas9 and Cas12a RNPs were assembled at a 1:1-5 Cas:guide ratio in TE buffer for 10 minutes at room temperature and mixed with cells immediate prior to electroporation. Post electroporation, cells were re-plated in supplemented HSC media for a further three days prior to genomic DNA harvest for analysis.

### T Cell Editing

T cells were cultured as described above. Dynabeads were removed using a DynaMag™-15 Magnet (Invitrogen), and cells washed in PBS and counted. For serum-free transduction prior to nucleofection, cells were washed again and resuspended in TexMACs media with pen-strep and IL-2 but without human serum. Nucleases were delivered into cells via electroporation using the Lonza 4D nucleofector, buffer P3, program EO-115—typically 1.5-3 million cells in the 1 ml cuvette, or 0.5-1 million the 20 μl cuvette.

### Determining Nuclease Efficiency Using TIDE/ICE Analysis

Genomic DNA was harvested from edited cells at day 5 post nucleofection (Qiagen). PCR amplicons were generated using the following primer pair for all nucleases (Fwd: TGGCCTCTGAGTAAACCGCA, Rev: AGCGAGGGATTGAGGCGAAA, product length: 718 bp, Tm: 69° C.) using Q5 polymerase (NEB). After PCR purification (Qiagen), amplicons were sanger sequenced (Eurofins genomics) using the forward primer. The resulting ab.1 file was the input to the online TIDE tool (Brinkman et al., 2014) (TALENs), or ICE software (Cas9/Cas12a, Synthego) which generated the % modification score.

### Assessment of Cutting Activity at Predicted Off-Target Loci

The top 10 most highly predicted loci for off-target nuclease activity of Cas9-3 and Cas12a-1 were identified by Benchling online software, while TALEN sites were predicted using PROGNOS (http://bao.rice.edu/cgi-bin/prognos/prognos.cgi) (Fine et al., 2014). PCR amplicons were designed to generate 150-200 bp with the expected cut site in the centre. DNA from male healthy donor T cells edited with each nuclease platform (alongside untreated controls) was extracted at day 3 post nucleofection (Qiagen) and used as a template for on-target and off-target PCR reactions. The amplicons were then prepared for Illumina next generation sequencing by performing end repair, adapter ligation and bar coding using the NEBNext® Ultra™ II DNA Library Prep Kit (NEB) according to the manufacturer's instructions. Libraries were quantified using the ddPCR™ Library Quantification Kit for Illumina TruSeq (Biorad), before sequencing using MiSeq Reagent Kit v2, 500 cycles on an Illumina MiSeq platform (Illumina). The generated paired-end reads were processed using the command line version of the CRISPResso2 pipeline (Clement et al., 2019), obtained editing frequencies were compared to the untreated control samples using Fisher's exact test. *, **, and *** indicate P<0.05, P<0.01, P<0.001, respectively.

### AAV6 Production and Purification

An AAV2 genome plasmid was kindly provided by Professor Amit Nathwani (UCL), and the pDGM6 (RRID:Addgene_110660) AAV6 packaging plasmid by the Russel lab (University of Washington). Homology arms were amplified from healthy control genomic DNA, both right and left 850 bp long in all donor constructs. Constructs were cloned using HiFi assembly (NEB), allowing the left homology arm sequence to run directly into a codon optimised SAP cDNA without restriction enzyme sequences. SH2D1A 3′UTR sequences were also amplified from genomic DNA, all other elements were amplified from previously described lentiviral plasmids (Rivat et al., 2013; Panchal et al., 2018b).

AAV6 particles were produced in HEK293T cells via co-transfection of pDGM6 and the HDR genome plasmid using polyethylenimine (PEI, Sigma-Aldrich) 24 hours after plating in complete DMEM media (DMEM (Gibco) 10% FBS, 1% penstrep). Media was replaced after 4 hours and replaced again after 24 hours with 2% DMEM. After a further 48 hours, the cells were released into the media by scraping, before centrifuging to separate cell pellet and culture supernatant for processing separately. AAV6 particles were precipitated from cell media using ammonium sulphate on ice for 30 min before collection by centrifugation and resuspended in TD buffer (1×PBS, 1 mM MgCl₂, 2.5 mM KCl). The cell pellet was resuspended in TD and freeze/thaw 4× in the presence of 0.5% deoxycholic acid (VWR), before centrifugation and harvesting the supernatant. Both fractions were incubated with benzonase 50 U/ml (Novagen) before combining prior to iodixanol-gradient centrifugation. AAV6 particles were harvested from the 40%-60% gradient interface and stored at 4° C. Titration was performed using the QuickTiter™ AAV Quantitation Kit (Cell Biolabs).

### Detection of SAP Protein Expression by Intracellular Staining and Flow Cytometry

Intracellular staining of SAP can be performed using the IntraPrep Permeabilizaton Reagent (Beckman). The primary antibody was either mouse anti-human SH2D1A antibody clone 1C9 ((Abnova Cat #H00004068-M01, RRID:AB_425532), or isotype control (Novus Cat #NB110-7082, RRID:AB_790752). The secondary was Goat anti-mouse polyclonal immunoglobulins RPE Goat F(ab′)2 (Dako).

### Statistical Analysis

Statistical analysis was performed with Graphpad Prism 9 software.

Data is presented as the mean±SEM or SD as denoted in the figure legend. Statistical analysis was performed using Prism 9 software (Graphpad Software Inc), details of statistical tests used, including all p values are indicated in the relevant figure legend.

Supplementary Table 1: Sequences

| SEQ ID | | | |
|---|---|---|---|
| NO: | Name | Type | Sequence |
| 1 | Sense- | DNA | ATGGACGCAGTGGCTGTGTATCATGGCAAAATCAGCA |
| | Exon 1 | | GGGAAACCGGCGAGAAGCTCCTGCTTGCCACTGGGC |
| | of | | TGGATGGCAGCTATTTGCTGAGGGACAGCGAGAGCGT |
| | human | | GCCAGGCGTGTACTGCCTATGTGTGCT |
| | SH2D1A | | |
| 2 | Anti- | DNA | AGCACACATAGGCAGTACACGCCTGGCACGCTCTCGC |
| | sense- | | TGTCCCTCAGCAAATAGCTGCCATCCAGCCCAGTGGC |
| | Exon 1 | | AAGCAGGAGCTTCTCGCCGGTTTCCCTGCT |
| | of | | GATTTTGCCATGATACACAGCCACTGCGTCCAT |
| | human | | |
| | SH2D1A | | |
| 3 | Portion | DNA | ATGGACGCAGTGGCTGTGTATCATGG |
| | of Exon | | |
| | 1 of | | |
| | human | | |
| | SH2D1A- | | |
| | Sense | | |
| 4 | Portion | DNA | CCATGATACACAGCCACTGCGTCCAT |
| | of Exon | | |
| | 1 of | | |
| | human | | |
| | SH2D1A | | |
| | Anti- | | |
| | sense | | |

| | | | | | | | Target DNA |
|---|---|---|---|---|---|---|---|
| | | | | | | | sequence |
| | | | | | | | (upstream or |
| SEQ ID | | | | Part of Guide | | | downstream of |
| NO: | Name | PAM | Type | RNA Sequence | SEQ ID NO: | Type | PAM) |
| 5 | Cas9-1 | GG | RNA | GUAUCAUGGCA | 30 | DNA | GTATCATGGC |
| | | G | | AAAUCAGCA | | | AAAATCAGCA |
| 6 | Cas9-1_{ι} | GG | RNA | UCAUGGCAAAAUCAG | 31 | DNA | TCATGGCAAAATCA |
| | | G | | CA | | | GCA |
| 7 | Cas9-2 | TGG | RNA | ACACAUAGGCAGUAC | 32 | DNA | ACACATAGGCAGTA |
| | | | | ACGCC | | | CACGCC |
| 8 | Cas9-2_{ι} | TGG | RNA | CAUAGGCAGUACACG | 33 | DNA | CATAGGCAGTACAC |
| | | | | CC | | | GCC |
| 9 | Cas9-3 | TGG | RNA | AUACACAGCCACUGC | 34 | DNA | ATACACAGCCACTG |
| | | | | GUCCA | | | CGTCCA |
| 10 | Cas9-3_{ι} | TGG | RNA | CACAGCCACUGCGUC | 35 | DNA | CACAGCCACTGCGT |
| | | | | CA | | | CCA |
| 11 | Cas9-4 | TGG | RNA | GACGCAGUGGCUGU | 36 | DNA | GACGCAGTGGCTGT |
| | | | | GUAUCA | | | GTATCA |
| 12 | Cas9-4_{ι} | TGG | RNA | GCAGUGGCUGUGUAU | 37 | DNA | GCAGTGGCTGTGTA |
| | | | | CA | | | TCA |
| 13 | Cas12a- | TTT | RNA | CCAUGAUACACAGCC | 38 | DNA | CCATGATACACAGC |
| | 1 | G | | ACUGCG | | | CACTGCG |
| 14 | Cas12a- | TTT | RNA | CCAUGAUACACAGCC | 39 | DNA | CCATGATACACAGC |
| | 1_{ι} | G | | AC | | | CAC |
| 15 | Cas12a- | TTT | RNA | CCUGCUGAUUUUGCC | 40 | DNA | CCTGCTGATTTTGC |
| | 2 | C | | AUGAUA | | | CATGATA |
| 16 | Cas12a- | TTT | RNA | CCUGCUGAUUUUGCC | 41 | DNA | CCTGCTGATTTTGC |
| | 2_{ι} | C | | AU | | | CAT |
| 17 | Cas12a- | TTT | RNA | CUGAGGGACAGCGAG | 42 | DNA | CTGAGGGACAGCGA |
| | 3 | G | | AGCGUG | | | GAGCGTG |
| 18 | Cas12a- | TTT | RNA | CUGAGGGACAGCGAG | 43 | DNA | CTGAGGGACAGCGA |
| | 3_{ι} | G | | AG | | | GAG |

-coSAP DNA
SEQ ID NO: 19
ATGGATGCCGTGGCCGTGTACCACGGCAAGATCAGCAGAGAGACAGGCGAGAA
GCTGCTGCTGGCCACCGGCCTGGACGGCAGCTACCTGCTGAGGGACAGCGAG
AGCGTGCCCGGCGTGTACTGCCTGTGCGTGCTGTATCACGGCTACATCTACAC
CTACCGGGTGTCCCAGACCGAGACAGGCTCTTGGAGCGCCGAGACAGCCCCT
GGCGTGCACAAGCGGTACTTCCGGAAGATCAAGAACCTGATCAGCGCCTTCCA
GAAGCCCGACCAGGGCATCGTGATCCCCCTGCAGTACCCCGTGGAAAAGAAGT
CCAGCGCCAGAAGCACCCAGGGCACCACCGGAATCCGCGAGGACCCCGACGT
GTGCCTGAAGGCCCCCTGA
-GFP
SEQ ID NO: 20
ATGGTGAGCAAGGGCGAGGAGCTGTTCACCGGGGTGGTGCCCATCCTGGTCG
AGCTGGACGGCGACGTAAACGGCCACAAGTTCAGCGTGTCCGGCGAGGGCGA
GGGCGATGCCACCTACGGCAAGCTGACCCTGAAGTTCATCTGCACCACCGGCA
AGCTGCCCGTGCCCTGGCCCACCCTCGTGACCACCCTGACCTACGGCGTGCAG
TGCTTCAGCCGCTACCCCGACCACATGAAGCAGCACGACTTCTTCAAGTCCGCC
ATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGC
ATCGAGCTGAAGGGCATCGACTTCAAGGAGGACGGCAACATCCTGGGGCACAA
GCTGGAGTACAACTACAACAGCCACAACGTCTATATCATGGCCGACAAGCAGAA
GAACGGCATCAAGGTGAACTTCAAGATCCGCCACAACATCGAGGACGGCAGCG
TGCAGCTCGCCGACCACTACCAGCAGAACACCCCCATCGGCGACGGCCCCGT
GCTGCTGCCCGACAACCACTACCTGAGCACCCAGTCCGCCCTGAGCAAAGACC
CCAACGAGAAGCGCGATCACATGGTCCTGCTGGAGTTCGTGACCGCCGCCGG
GATCACTCTCGGCATGGACGAGCTGTACAAGTAA
-HAL
SEQ ID NO: 21
AGGCAAAGCACCAGTTAAATGATTCACAGTCTCATCTAAAGCCCATCAAATAGAT
ACATCTGGAACATCTTTGGATGTACATTGTGACTTTTGTATCTCAACTAGCACTTT
GGGACCTTGGACAAGGTGTTAGGAAAGAAATCAATATTTGCTCAGGATCAAGAC
TTCTCAAGATGACTGCGTGAGGTAGAAGGAAGAGGGAGAAGAAGAACTTGCCC
AGAGTTCACTGTTCATCTACTATGTCTGATTCTGTAGGAGTCTTCATGGCCATGT
TCAGTAATGATTGCATACTGACAGTACTGATAAAACTATCAGTGTTTTGTTCTCA
GAGCTCTTGGTCCTTTCATTTTGTCAGTGTGTTTTTGCCCTACGTAGTGGGTCCA
CATACCAACAGAGACATGTACAACTTGCCTCTATCATCAACTCCTTATGGGCTAG
TTGAGCTAAGTTATTCCTGGTGGCCTCTGAGTAAACCGCAAGAACAGAAGCATT
ACTAAGCATCCCCGTCTGAGTGCAAGGGTGTGTGTTGGCAGTACAGCCCCAAT
CTTGCAAAATCCTTCTTCCAATGTTCCTCCCCTCTCTGTATGAACCCTGTGTTGG
GGGGCAGAAGATGGAAGCCCTTGGCAAGCTCGATCGAACCAAGCTACTAAATT
GCTGAGCTCGTTTTAACTGAAGTGTGAGAAGGAGGTTTAAGGCAAGTAGACAAC
ATCCTGTTGTTGGGGTGCTTCTCTCTTTTTTGCACATCTGGCTGAACTGGGAGTC
AGGTGGTTGACTTGTGCCTGGCTGCAGTAGCAGCGGCATCTCCCTTGCACAGT
TCTCCTCCTCGGCCTGCCCAAGAGTCCACCAGGCC
-HAR
SEQ ID NO: 22
TGGACGCAGTGGCTGTGTATCATGGCAAAATCAGCAGGGAAACCGGCGAGAAG
CTCCTGCTTGCCACTGGGCTGGATGGCAGCTATTTGCTGAGGGACAGCGAGAG
CGTGCCAGGCGTGTACTGCCTATGTGTGCTGTGAGTATGATACGGTGGACATG
GGCCTGCTGAGGGTGTGGGCGGTGGGCAACAGCAGCTGGGGCCAGGGTGGA
GGCCGAGGCAGGCAGGGGCGCCGGCGTTAGCAGCTCGCCGACGCCTCCTCC
GTGGCCCACCCTCAAGTCCAGCCCAGGGCCGTGGTGGAACACACTTTCGCCTC
AATCCCTCGCTGGGGATTTCATTCCCTGGGCCTCCTGCTCTCCAGAAGGCCCTC
ACCAGAGCCTTTTGGGGCAGCTGCAGAGAATGCCATTTCGGGTGGTGAAATGT
GGTCCACAGTTTACCTTCGCTCCGGGAAAGACCCAGGCTCCCCACTTCCATTTT
GACCCCTATCGGGTGGCGCCCTGCGAGCCTTACTCTAGCTCTCTTTAAACGACT
TTTGAAGTGTCATCCCAAAGGAAGAGCATGACATTCAGAGAGAGGAGGCACATT
AAGAAACAGAGTCAATTACTTTAAGAATGCAATGCCAGTTAGGTCGAGATGCTG
AGAGAGGAGGGGGATGTGTGTCAGTAAAATCACCTCTCTGGATCTGCTGATGTA
GCATCCTTTAGAAAAGTAGTGGGGGGTGGGGAGGGGCAGGGGATTTCAGAAAT
AGGACCCTTCTTGATAAACGTTAGGACTCACAATAGTCTTAAAGGGAATGGAGG
GAAAACACCCTTGTTATGATTCAGCTTTGCCATTTGCCTAGGCATGTCTCAT
-SV40pA
SEQ ID NO: 23
TTGCTTTATTTGTGAAATTTGTGATGCTATTGCTTTATTTGTAACCATTATAAGCT
GCAATAAACAAGTTAACAACAACAATTGCATTCATTTTATGTTTCAGGTTCAGGG
GGAGGTGTGGGAGGTTTTTTAAA
SEQ ID NO 24: bGHpA
CTGTGCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAAATTG
CATCGCATTGTCTGAGTAGGTGTCATTCTATTCTGGGGGGTGGGGTGGGGCAG
GACAGCAAGGGGGAGGATTGGGAAGACAATAGCAGGCATGCTGGGGATGCGG
TGGGCTCTATGGGT
-SAP UTR
SEQ ID NO: 25
AGAAAAATAAAACACCTTGTACTTTATTTTCTATAATTTAAATATATGCTAAGTCTTATATA
TTGTAGATAATACAGTTCGGTGAGCTACAAATGCATTTCTAAAGCCATTGTAGTCCTGTA
ATGGAAGCATCTAGCATGTCGTCAAAGCTGAAATGGACTTTTGTACATAGTGAGGAGCT
TTGAAACGAGGATTGGGAAAAAGTAATTCCGTAGGTTATTTTCAGTTATTATATTTACAAA
TGGGAAACAAAAGGATAATGAATACTTTATAAAGGATTAATGTCAATTCTTGCCAAATATA
AATAAAAATAATCCTCAGTTTTTGTGAAAAGCTCCATTTTTAGTGAAATATTATTTTATAGC
TACTAATTTTAAAATGTCTTGCTTGATTGTATGGTGGGAAGTTGGCTGGTGTCCCTTGTC
TTTGCCAAGTTCTCCACTAGCTATGGTGTCATAGGCTCTTTTGGGATTTTTGAAGCTGTA
TACTGTGTGCTAAAACAAGCACTAAACAAAGAGTGAAGGATTTATGTTTAATTCTGAAAG
CAACCTTCTTGCCTAGTGTTCTGATATTGGACAGTAAAATCCACAGACCAACCTGGAGTT
GAAAATCTTATAATTTAAAATATGCTCTAAACATGTTTATCGTATTTGATGCTACAGGATTT
GAAATTGTATTACAAATCCAATGAAATGAGTTTTTCTTTTCATTTACCTCTGCCCCAGTTG
TTTCTACTACATGGAAGACCTCATTTTGAAGGGAAATTTCAGCAGCTGCAGCTCATGAGT
AACTGATTTGTAACAAGCCTCCTTTTAAAGTAACCCTACAAAACCACTGGAAAGTTTATG
GTTGTATTATTTTTTAAAAAAATTCCAAGTGATTGAAACCTACACGAGATACAGAATTTTA
TGCGGCATTTTCTTCTCACATTTATATTTTTGTGATTTTGTGATTGATTATATGTCACTTTG
CTACAGGGCTCACAGAATTCATTCACTCAACAAACATAATAGGGCGCTGAGGGCATAGA
AGTAAAAACACCTGGTCCCTGCTCTCAGTTCACTGTCTTGTTGGACGAGAAAACAATAAC
GATAAAAGACAGTGAAAGAAAATAACGATAAAAGACAGTGAAAGAAAATAACAATAAAAG
ACAAGGAAAAAATAACAATGAAAGTTGATAAGTACATGATAAGCGAGGTTCCCCGTGTG
TAGGTAGATCTGGTCTTTAGAGGCAGATAGATAGGTCAGTGCAAATACTCTGGTCCATG
GGCCATATGAAAAGGCTAAGCTTCACTGTAAAATAATAACTGGGAATTCTGGATTGTGTA
TGGGTGTTGGTGAACTTGGTTTTAATTAGTGAACTGCTGAGAGACAGAGCTATTCTCCAT
GTACTGGCAAGACCTGATTTCTGAGCATTTAATATGGATGCCGTGGGAGTACAAAAGTG
GAGTGTGGCCTGAGTAATGCATTATGGGTGGTTTACCATTTCTTGAGGTAAAAGCATCA
CATGAACTTGTAAAGGAATTTAAAAATCCTACTTTCATAATAAGTTGCATAGGTTTAATAA
TTTTTAATTATATGGCTTGAGTTTAAATTGTAATAGGCGTAACTAATTTTAACTCTATAATG
TGTTCATTCTGGAATAATCCTAAACATATGAATTATGTTTGCATGTTCACTTCCAAGAGCC
TTTTTTTGAAAAAAAGCTTTTTTTGAATCATCAAGTCTTTCACATTTAAATAAAGTGTTTGA
AAGCTTTATTTA
-WPREmut6
SEQ ID NO: 26
AATCAACCTCTGGATTACAAAATTTGTGAAAGATTGACTGGTATTCTTAACTATGT
TGCTCCTTTTACGCTATGTGGATACGCTGCTTTAATGCCTTTGTATCATGCTATT
GCTTCCCGTATGGCTTTCATTTTCTCCTCCTTGTATAAATCCTGGTTGCTGTCTC
TTTATGAGGAGTTGTGGCCCGTTGTCAGGCAACGTGGCGTGGTGTGCACTGTG
TTTGCTGACGCAACCCCCACTGGTTGGGGCATTGCCACCACCTGTCAGCTCCTT
TCCGGGACTTTCGCTTTCCCCCTCCCTATTGCCACGGCGGAACTCATCGCCGC
CTGCCTTGCCCGCTGCTGGACAGGGGCTCGGCTGTTGGGCACTGACAATTCCG
TGGTGTTGTCGGGGAAATCATCGTCCTTTCCTTGGCTGCTCGCCTGTGTTGCCA
CCTGGATTCTGCGCGGGACGTCCTTCTGCTACGTCCCTTCGGCCCTCAATCCA
GCGGACCTTCCTTCCCGCGGCCTGCTGCCGGCTCTGCGGCCTCTTCCGCGTCT
TCGCCTTCGCCCTCAGACGAGTCGGATCTCCCTTTGGGCCGCCTCCCCGCCTG
G7bc,
SEQ ID NO: 27
AGGCAAAGCACCAGTTAAATGATTCACAGTCTCATCTAAAGCCCATCAAATAGAT
ACATCTGGAACATCTTTGGATGTACATTGTGACTTTTGTATCTCAACTAGCACTTT
GGGACCTTGGACAAGGTGTTAGGAAAGAAATCAATATTTGCTCAGGATCAAGAC
TTCTCAAGATGACTGCGTGAGGTAGAAGGAAGAGGGAGAAGAAGAACTTGCCC
AGAGTTCACTGTTCATCTACTATGTCTGATTCTGTAGGAGTCTTCATGGCCATGT
TCAGTAATGATTGCATACTGACAGTACTGATAAAACTATCAGTGTTTTGTTCTCA
GAGCTCTTGGTCCTTTCATTTTGTCAGTGTGTTTTTGCCCTACGTAGTGGGTCCA
CATACCAACAGAGACATGTACAACTTGCCTCTATCATCAACTCCTTATGGGCTAG
TTGAGCTAAGTTATTCCTGGTGGCCTCTGAGTAAACCGCAAGAACAGAAGCATT
ACTAAGCATCCCCGTCTGAGTGCAAGGGTGTGTGTTGGCAGTACAGCCCCAAT
CTTGCAAAATCCTTCTTCCAATGTTCCTCCCCTCTCTGTATGAACCCTGTGTTGG
GGGGCAGAAGATGGAAGCCCTTGGCAAGCTCGATCGAACCAAGCTACTAAATT
GCTGAGCTCGTTTTAACTGAAGTGTGAGAAGGAGGTTTAAGGCAAGTAGACAAC
ATCCTGTTGTTGGGGTGCTTCTCTCTTTTTTGCACATCTGGCTGAACTGGGAGTC
AGGTGGTTGACTTGTGCCTGGCTGCAGTAGCAGCGGCATCTCCCTTGCACAGT
TCTCCTCCTCGGCCTGCCCAAGAGTCCACCAGGCCTGACTGTGCCTTCTAGTTG
CCAGCCATCTGTTGTTTGCCCCTCCCCCGTGCCTTCCTTGACCCTGGAAGGTGC
CACTCCCACTGTCCTTTCCTAATAAAATGAGGAAATTGCATCGCATTGTCTGAGT
AGGTGTCATTCTATTCTGGGGGGTGGGGGGGGCAGGACAGCAAGGGGGAGG
ATTGGGAAGACAATAGCAGGCATGCTGGGGATGCGGTGGGCTCTATGGGTACG
CGTCTCCGGTGCCCGTCAGTGGGCAGAGCGCACATCGCCCACAGTCCCCGAG
AAGTTGGGGGGAGGGGTCGGCAATTGAACCGGTGCCTAGAGAAGGTGGCGCG
GGGTAAACTGGGAAAGTGATGTCGTGTACTGGCTCCGCCTTTTTCCCGAGGGT
GGGGGAGAACCGTATATAAGTGCAGTAGTCGCCGTGAACGTTCTTTTTCGCAAC
GGGTTTGCCGCCAGAACACAGGTGTCGTGACGCGGGATCCGCCACCATGGTGA
GCAAGGGCGAGGAGCTGTTCACCGGGGTGGTGCCCATCCTGGTCGAGCTGGA
CGGCGACGTAAACGGCCACAAGTTCAGCGTGTCCGGCGAGGGCGAGGGCGAT
GCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCA
GCCGCTACCCCGACCACATGAAGCAGCACGACTTCTTCAAGTCCGCCATGCCC
GAAGGCTACGTCCAGGAGCGCACCATCTTCTTCAAGGACGACGGCAACTACAA
GACCCGCGCCGAGGTGAAGTTCGAGGGCGACACCCTGGTGAACCGCATCGAG
CTGAAGGGCATCGACTTCAAGGAGGACGGCAACATCCTGGGGCACAAGCTGGA
GTACAACTACAACAGCCACAACGTCTATATCATGGCCGACAAGCAGAAGAACGG
CATCAAGGTGAACTTCAAGATCCGCCACAACATCGAGGACGGCAGCGTGCAGC
TCGCCGACCACTACCAGCAGAACACCCCCATCGGCGACGGCCCCGTGCTGCTG
CCCGACAACCACTACCTGAGCACCCAGTCCGCCCTGAGCAAAGACCCCAACGA
GAAGCGCGATCACATGGTCCTGCTGGAGTTCGTGACCGCCGCCGGGATCACTC
TCGGCATGGACGAGCTGTACAAGTAAGTCGACAATCAACCTCTGGATTACAAAA
TTTGTGAAAGATTGACTGGTATTCTTAACTATGTTGCTCCTTTTACGCTATGTGGA
TACGCTGCTTTAATGCCTTTGTATCATGCTATTGCTTCCCGTATGGCTTTCATTTT
CTCCTCCTTGTATAAATCCTGGTTGCTGTCTCTTTATGAGGAGTTGTGGCCCGTT
GTCAGGCAACGTGGCGTGGTGTGCACTGTGTTTGCTGACGCAACCCCCACTGG
TTGGGGCATTGCCACCACCTGTCAGCTCCTTTCCGGGACTTTCGCTTTCCCCCT
CCCTATTGCCACGGCGGAACTCATCGCCGCCTGCCTTGCCCGCTGCTGGACAG
GGGCTCGGCTGTTGGGCACTGACAATTCCGTGGTGTTGTCGGGGAAATCATCG
TCCTTTCCTTGGCTGCTCGCCTGTGTTGCCACCTGGATTCTGCGCGGGACGTCC
TTCTGCTACGTCCCTTCGGCCCTCAATCCAGCGGACCTTCCTTCCCGCGGCCTG
CTGCCGGCTCTGCGGCCTCTTCCGCGTCTTCGCCTTCGCCCTCAGACGAGTCG
GATCTCCCTTTGGGCCGCCTCCCCGCCTGGCGGCCGCACTAGTTGCTTTATTTG
TGAAATTTGTGATGCTATTGCTTTATTTGTAACCATTATAAGCTGCAATAAACAAG
TTAACAACAACAATTGCATTCATTTTATGTTTCAGGTTCAGGGGGAGGTGTGGGA
GGTTTTTTAAATGGACGCAGTGGCTGTGTATCATGGCAAAATCAGCAGGGAAAC
CGGCGAGAAGCTCCTGCTTGCCACTGGGCTGGATGGCAGCTATTTGCTGAGGG
ACAGCGAGAGCGTGCCAGGCGTGTACTGCCTATGTGTGCTGTGAGTATGATAC
GGTGGACATGGGCCTGCTGAGGGTGTGGGCGGTGGGCAACAGCAGCTGGGGC
CAGGGTGGAGGCCGAGGCAGGCAGGGGCGCCGGCGTTAGCAGCTCGCCGAC
GCCTCCTCCGTGGCCCACCCTCAAGTCCAGCCCAGGGCCGTGGTGGAACACAC
TTTCGCCTCAATCCCTCGCTGGGGATTTCATTCCCTGGGCCTCCTGCTCTCCAG
AAGGCCCTCACCAGAGCCTTTTGGGGCAGCTGCAGAGAATGCCATTTCGGGTG
GTGAAATGTGGTCCACAGTTTACCTTCGCTCCGGGAAAGACCCAGGCTCCCCA
CTTCCATTTTGACCCCTATCGGGTGGCGCCCTGCGAGCCTTACTCTAGCTCTCT
TTAAACGACTTTTGAAGTGTCATCCCAAAGGAAGAGCATGACATTCAGAGAGAG
GAGGCACATTAAGAAACAGAGTCAATTACTTTAAGAATGCAATGCCAGTTAGGTC
GAGATGCTGAGAGAGGAGGGGGATGTGTGTCAGTAAAATCACCTCTCTGGATC
TGCTGATGTAGCATCCTTTAGAAAAGTAGTGGGGGGTGGGGAGGGGCAGGGGA
TTTCAGAAATAGGACCCTTCTTGATAAACGTTAGGACTCACAATAGTCTTAAAGG
GAATGGAGGGAAAACACCCTTGTTATGATTCAGCTTTGCCATTTGCCTAGGCAT
GTCTCAT
G15,
SEQ ID NO: 28
AGGCAAAGCACCAGTTAAATGATTCACAGTCTCATCTAAAGCCCATCAAATAGAT
ACATCTGGAACATCTTTGGATGTACATTGTGACTTTTGTATCTCAACTAGCACTTT
GGGACCTTGGACAAGGTGTTAGGAAAGAAATCAATATTTGCTCAGGATCAAGAC
TTCTCAAGATGACTGCGTGAGGTAGAAGGAAGAGGGAGAAGAAGAACTTGCCC
AGAGTTCACTGTTCATCTACTATGTCTGATTCTGTAGGAGTCTTCATGGCCATGT
TCAGTAATGATTGCATACTGACAGTACTGATAAAACTATCAGTGTTTTGTTCTCA
GAGCTCTTGGTCCTTTCATTTTGTCAGTGTGTTTTTGCCCTACGTAGTGGGTCCA
CATACCAACAGAGACATGTACAACTTGCCTCTATCATCAACTCCTTATGGGCTAG
TTGAGCTAAGTTATTCCTGGTGGCCTCTGAGTAAACCGCAAGAACAGAAGCATT
ACTAAGCATCCCCGTCTGAGTGCAAGGGTGTGTGTTGGCAGTACAGCCCCAAT
CTTGCAAAATCCTTCTTCCAATGTTCCTCCCCTCTCTGTATGAACCCTGTGTTGG
GGGGCAGAAGATGGAAGCCCTTGGCAAGCTCGATCGAACCAAGCTACTAAATT
GCTGAGCTCGTTTTAACTGAAGTGTGAGAAGGAGGTTTAAGGCAAGTAGACAAC
ATCCTGTTGTTGGGGTGCTTCTCTCTTTTTTGCACATCTGGCTGAACTGGGAGTC
AGGTGGTTGACTTGTGCCTGGCTGCAGTAGCAGCGGCATCTCCCTTGCACAGT
TCTCCTCCTCGGCCTGCCCAAGAGTCCACCAGGCCATGGATGCCGTGGCCGTG
TACCACGGCAAGATCAGCAGAGAGACAGGCGAGAAGCTGCTGCTGGCCACCG
GCCTGGACGGCAGCTACCTGCTGAGGGACAGCGAGAGCGTGCCCGGCGTGTA
CTGCCTGTGCGTGCTGTATCACGGCTACATCTACACCTACCGGGTGTCCCAGAC
CGAGACAGGCTCTTGGAGCGCCGAGACAGCCCCTGGCGTGCACAAGCGGTAC
TTCCGGAAGATCAAGAACCTGATCAGCGCCTTCCAGAAGCCCGACCAGGGCAT
CGTGATCCCCCTGCAGTACCCCGTGGAAAAGAAGTCCAGCGCCAGAAGCACCC
AGGGCACCACCGGAATCCGCGAGGACCCCGACGTGTGCCTGAAGGCCCCCGC
TACCAACTTTTCCCTGCTGAAACAGGCAGGCGATGTGGAGGAAAATCCTGGGC
CAATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGGGC
GAGGGCGATGCCACCTACGGCAAGCTGACCCTGAAGTTCATCTGCACCACCGG
CAAGCTGCCCGTGCCCTGGCCCACCCTCGTGACCACCCTGACCTACGGCGTGC
AGTGCTTCAGCCGCTACCCCGACCACATGAAGCAGCACGACTTCTTCAAGTCCG
CCATGCCCGAAGGCTACGTCCAGGAGCGCACCATCTTCTTCAAGGACGACGGC
AACTACAAGACCCGCGCCGAGGTGAAGTTCGAGGGCGACACCCTGGTGAACCG
CATCGAGCTGAAGGGCATCGACTTCAAGGAGGACGGCAACATCCTGGGGCACA
AGCTGGAGTACAACTACAACAGCCACAACGTCTATATCATGGCCGACAAGCAGA
AGAACGGCATCAAGGTGAACTTCAAGATCCGCCACAACATCGAGGACGGCAGC
GTGCAGCTCGCCGACCACTACCAGCAGAACACCCCCATCGGCGACGGCCCCG
TGCTGCTGCCCGACAACCACTACCTGAGCACCCAGTCCGCCCTGAGCAAAGAC
CCCAACGAGAAGCGCGATCACATGGTCCTGCTGGAGTTCGTGACCGCCGCCGG
GATCACTCTCGGCATGGACGAGCTGTACAAGTAAGTCGACAATCAACCTCTGGA
TTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGC
TTTCATTTTCTCCTCCTTGTATAAATCCTGGTTGCTGTCTCTTTATGAGGAGTTGT
GGCCCGTTGTCAGGCAACGTGGCGTGGTGTGCACTGTGTTTGCTGACGCAACC
CCCACTGGTTGGGGCATTGCCACCACCTGTCAGCTCCTTTCCGGGACTTTCGCT
TTCCCCCTCCCTATTGCCACGGCGGAACTCATCGCCGCCTGCCTTGCCCGCTG
CTGGACAGGGGCTCGGCTGTTGGGCACTGACAATTCCGTGGTGTTGTCGGGGA
AATCATCGTCCTTTCCTTGGCTGCTCGCCTGTGTTGCCACCTGGATTCTGCGCG
GGACGTCCTTCTGCTACGTCCCTTCGGCCCTCAATCCAGCGGACCTTCCTTCCC
GCGGCCTGCTGCCGGCTCTGCGGCCTCTTCCGCGTCTTCGCCTTCGCCCTCAG
ACGAGTCGGATCTCCCTTTGGGCCGCCTCCCCGCCTGGCGGCCGCACTAGTTG
CTTTATTTGTGAAATTTGTGATGCTATTGCTTTATTTGTAACCATTATAAGCTGCA
ATAAACAAGTTAACAACAACAATTGCATTCATTTTATGTTTCAGGTTCAGGGGGA
GGTGTGGGAGGTTTTTTAAATGGACGCAGTGGCTGTGTATCATGGCAAAATCAG
CAGGGAAACCGGCGAGAAGCTCCTGCTTGCCACTGGGCTGGATGGCAGCTATT
TGCTGAGGGACAGCGAGAGCGTGCCAGGCGTGTACTGCCTATGTGTGCTGTGA
GTATGATACGGTGGACATGGGCCTGCTGAGGGTGTGGGCGGTGGGCAACAGC
AGCTGGGGCCAGGGTGGAGGCCGAGGCAGGCAGGGGCGCCGGCGTTAGCAG
CTCGCCGACGCCTCCTCCGTGGCCCACCCTCAAGTCCAGCCCAGGGCCGTGG
TGGAACACACTTTCGCCTCAATCCCTCGCTGGGGATTTCATTCCCTGGGCCTCC
TGCTCTCCAGAAGGCCCTCACCAGAGCCTTTTGGGGCAGCTGCAGAGAATGCC
ATTTCGGGTGGTGAAATGTGGTCCACAGTTTACCTTCGCTCCGGGAAAGACCCA
GGCTCCCCACTTCCATTTTGACCCCTATCGGGTGGCGCCCTGCGAGCCTTACTC
TAGCTCTCTTTAAACGACTTTTGAAGTGTCATCCCAAAGGAAGAGCATGACATTC
AGAGAGAGGAGGCACATTAAGAAACAGAGTCAATTACTTTAAGAATGCAATGCC
AGTTAGGTCGAGATGCTGAGAGAGGAGGGGGATGTGTGTCAGTAAAATCACCT
CTCTGGATCTGCTGATGTAGCATCCTTTAGAAAAGTAGTGGGGGGTGGGGAGG
GGCAGGGGATTTCAGAAATAGGACCCTTCTTGATAAACGTTAGGACTCACAATA
GTCTTAAAGGGAATGGAGGGAAAACACCCTTGTTATGATTCAGCTTTGCCATTTG
CCTAGGCATGTCTCAT
-G16
SEQ ID NO: 29
AGGCAAAGCACCAGTTAAATGATTCACAGTCTCATCTAAAGCCCATCAAATAGAT
ACATCTGGAACATCTTTGGATGTACATTGTGACTTTTGTATCTCAACTAGCACTTT
GGGACCTTGGACAAGGTGTTAGGAAAGAAATCAATATTTGCTCAGGATCAAGAC
TTCTCAAGATGACTGCGTGAGGTAGAAGGAAGAGGGAGAAGAAGAACTTGCCC
AGAGTTCACTGTTCATCTACTATGTCTGATTCTGTAGGAGTCTTCATGGCCATGT
TCAGTAATGATTGCATACTGACAGTACTGATAAAACTATCAGTGTTTTGTTCTCA
GAGCTCTTGGTCCTTTCATTTTGTCAGTGTGTTTTTGCCCTACGTAGTGGGTCCA
CATACCAACAGAGACATGTACAACTTGCCTCTATCATCAACTCCTTATGGGCTAG
TTGAGCTAAGTTATTCCTGGTGGCCTCTGAGTAAACCGCAAGAACAGAAGCATT
ACTAAGCATCCCCGTCTGAGTGCAAGGGTGTGTGTTGGCAGTACAGCCCCAAT
CTTGCAAAATCCTTCTTCCAATGTTCCTCCCCTCTCTGTATGAACCCTGTGTTGG
GGGGCAGAAGATGGAAGCCCTTGGCAAGCTCGATCGAACCAAGCTACTAAATT
GCTGAGCTCGTTTTAACTGAAGTGTGAGAAGGAGGTTTAAGGCAAGTAGACAAC
ATCCTGTTGTTGGGGTGCTTCTCTCTTTTTTGCACATCTGGCTGAACTGGGAGTC
AGGTGGTTGACTTGTGCCTGGCTGCAGTAGCAGCGGCATCTCCCTTGCACAGT
TCTCCTCCTCGGCCTGCCCAAGAGTCCACCAGGCCATGGATGCCGTGGCCGTG
TACCACGGCAAGATCAGCAGAGAGACAGGCGAGAAGCTGCTGCTGGCCACCG
GCCTGGACGGCAGCTACCTGCTGAGGGACAGCGAGAGCGTGCCCGGCGTGTA
CTGCCTGTGCGTGCTGTATCACGGCTACATCTACACCTACCGGGTGTCCCAGAC
CGAGACAGGCTCTTGGAGCGCCGAGACAGCCCCTGGCGTGCACAAGCGGTAC
TTCCGGAAGATCAAGAACCTGATCAGCGCCTTCCAGAAGCCCGACCAGGGCAT
CGTGATCCCCCTGCAGTACCCCGTGGAAAAGAAGTCCAGCGCCAGAAGCACCC
AGGGCACCACCGGAATCCGCGAGGACCCCGACGTGTGCCTGAAGGCCCCCGC
TACCAACTTTTCCCTGCTGAAACAGGCAGGCGATGTGGAGGAAAATCCTGGGC
CAATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGGGC
GAGGGCGATGCCACCTACGGCAAGCTGACCCTGAAGTTCATCTGCACCACCGG
CAAGCTGCCCGTGCCCTGGCCCACCCTCGTGACCACCCTGACCTACGGCGTGC
AGTGCTTCAGCCGCTACCCCGACCACATGAAGCAGCACGACTTCTTCAAGTCCG
CCATGCCCGAAGGCTACGTCCAGGAGCGCACCATCTTCTTCAAGGACGACGGC
AACTACAAGACCCGCGCCGAGGTGAAGTTCGAGGGCGACACCCTGGTGAACCG
CATCGAGCTGAAGGGCATCGACTTCAAGGAGGACGGCAACATCCTGGGGCACA
AGCTGGAGTACAACTACAACAGCCACAACGTCTATATCATGGCCGACAAGCAGA
AGAACGGCATCAAGGTGAACTTCAAGATCCGCCACAACATCGAGGACGGCAGC
GTGCAGCTCGCCGACCACTACCAGCAGAACACCCCCATCGGCGACGGCCCCG
TGCTGCTGCCCGACAACCACTACCTGAGCACCCAGTCCGCCCTGAGCAAAGAC
CCCAACGAGAAGCGCGATCACATGGTCCTGCTGGAGTTCGTGACCGCCGCCGG
GATCACTCTCGGCATGGACGAGCTGTACAAGTAAAGAAAAATAAAACACCTTGT
ACTTTATTTTCTATAATTTAAATATATGCTAAGTCTTATATATTGTAGATAATACAG
TTCGGTGAGCTACAAATGCATTTCTAAAGCCATTGTAGTCCTGTAATGGAAGCAT
CTAGCATGTCGTCAAAGCTGAAATGGACTTTTGTACATAGTGAGGAGCTTTGAAA
CGAGGATTGGGAAAAAGTAATTCCGTAGGTTATTTTCAGTTATTATATTTACAAAT
GGGAAACAAAAGGATAATGAATACTTTATAAAGGATTAATGTCAATTCTTGCCAA
ATATAAATAAAAATAATCCTCAGTTTTTGTGAAAAGCTCCATTTTTAGTGAAATATT
ATTTTATAGCTACTAATTTTAAAATGTCTTGCTTGATTGTATGGTGGGAAGTTGGC
TGGTGTCCCTTGTCTTTGCCAAGTTCTCCACTAGCTATGGTGTCATAGGCTCTTT
TGGGATTTTTGAAGCTGTATACTGTGTGCTAAAACAAGCACTAAACAAAGAGTGA
AGGATTTATGTTTAATTCTGAAAGCAACCTTCTTGCCTAGTGTTCTGATATTGGA
CAGTAAAATCCACAGACCAACCTGGAGTTGAAAATCTTATAATTTAAAATATGCT
CTAAACATGTTTATCGTATTTGATGCTACAGGATTTGAAATTGTATTACAAATCCA
ATGAAATGAGTTTTTCTTTTCATTTACCTCTGCCCCAGTIGTTTCTACTACATGGA
AGACCTCATTTTGAAGGGAAATTTCAGCAGCTGCAGCTCATGAGTAACTGATTTG
TAACAAGCCTCCTTTTAAAGTAACCCTACAAAACCACTGGAAAGTTTATGGTTGT
ATTATTTTTTAAAAAAATTCCAAGTGATTGAAACCTACACGAGATACAGAATTTTA
TGCGGCATTTTCTTCTCACATTTATATTTTTGTGATTTTGTGATTGATTATATGTCA
CTTTGCTACAGGGCTCACAGAATTCATTCACTCAACAAACATAATAGGGCGCTG
AGGGCATAGAAGTAAAAACACCTGGTCCCTGCTCTCAGTTCACTGTCTTGTTGG
ACGAGAAAACAATAACGATAAAAGACAGTGAAAGAAAATAACGATAAAAGACAGT
GAAAGAAAATAACAATAAAAGACAAGGAAAAAATAACAATGAAAGTTGATAAGTA
CATGATAAGCGAGGTTCCCCGTGTGTAGGTAGATCTGGTCTTTAGAGGCAGATA
GATAGGTCAGTGCAAATACTCTGGTCCATGGGCCATATGAAAAGGCTAAGCTTC
ACTGTAAAATAATAACTGGGAATTCTGGATTGTGTATGGGTGTTGGTGAACTTGG
TTTTAATTAGTGAACTGCTGAGAGACAGAGCTATTCTCCATGTACTGGCAAGACC
TGATTTCTGAGCATTTAATATGGATGCCGTGGGAGTACAAAAGTGGAGTGTGGC
CTGAGTAATGCATTATGGGTGGTTTACCATTTCTTGAGGTAAAAGCATCACATGA
ACTTGTAAAGGAATTTAAAAATCCTACTTTCATAATAAGTTGCATAGGTTTAATAA
TTTTTAATTATATGGCTTGAGTTTAAATTGTAATAGGCGTAACTAATTTTAACTCTA
TAATGTGTTCATTCTGGAATAATCCTAAACATATGAATTATGTTTGCATGTTCACT
TCCAAGAGCCTTTTTTTGAAAAAAAGCTTTTTTTGAATCATCAAGTCTTTCACATT
TAAATAAAGTGTTTGAAAGCTTTATTTATGGACGCAGTGGCTGTGTATCATGGCA
AAATCAGCAGGGAAACCGGCGAGAAGCTCCTGCTTGCCACTGGGCTGGATGGC
AGCTATTTGCTGAGGGACAGCGAGAGCGTGCCAGGCGTGTACTGCCTATGTGT
GCTGTGAGTATGATACGGTGGACATGGGCCTGCTGAGGGTGTGGGCGGTGGG
CAACAGCAGCTGGGGCCAGGGTGGAGGCCGAGGCAGGCAGGGGCGCCGGCG
TTAGCAGCTCGCCGACGCCTCCTCCGTGGCCCACCCTCAAGTCCAGCCCAGGG
CCGTGGTGGAACACACTTTCGCCTCAATCCCTCGCTGGGGATTTCATTCCCTGG
GCCTCCTGCTCTCCAGAAGGCCCTCACCAGAGCCTTTTGGGGCAGCTGCAGAG
AATGCCATTTCGGGTGGTGAAATGTGGTCCACAGTTTACCTTCGCTCCGGGAAA
GACCCAGGCTCCCCACTTCCATTTTGACCCCTATCGGGTGGCGCCCTGCGAGC
CTTACTCTAGCTCTCTTTAAACGACTTTTGAAGTGTCATCCCAAAGGAAGAGCAT
GACATTCAGAGAGAGGAGGCACATTAAGAAACAGAGTCAATTACTTTAAGAATG
CAATGCCAGTTAGGTCGAGATGCTGAGAGAGGAGGGGGATGTGTGTCAGTAAA
ATCACCTCTCTGGATCTGCTGATGTAGCATCCTTTAGAAAAGTAGTGGGGGGTG
GGGAGGGGCAGGGGATTTCAGAAATAGGACCCTTCTTGATAAACGTTAGGACT
CACAATAGTCTTAAAGGGAATGGAGGGAAAACACCCTTGTTATGATTCAGCTTTG
CCATTTGCCTAGGCATGTCTCAT

Supplementary Table 2: Primer sequences for Cas12a-1, Cas9-3 and TALEN on and off target PCR amplicons. Primers are given in the 5′-3′ orientation. For Cas12a-1, Cas9-3, the off-target positions are highlighted in bold, TALEN mismatches are denoted by a lowercase letter. For TALEN orientation, the letter denotes Left (L) or right (R) TALEN arm, the number indicating the spacer difference. TALEN off targets were identified in silico by PROGNOS, Cas9-3 and Cas12a-1 by Benchling.

| Cas12a- | | | | | Forward | Reverse |
|---|---|---|---|---|---|---|
| 1 | Binding sequence | PAM | | Gene | primer | primer |
| ON | CCATGATACACAGCCA | TTTG | chrX:−124346651 | SH2D1A | CTGGGAGTCA | CTCGCTGTCCCTC |
| | CTGCG (SEQ ID NO: 38) | | | | GGTGGTTGAC | AGCAAAT (SEQ ID |
| | | | | | (SEQ ID NO: 87) | NO: 88) |
| OT1 | CCATGAAACACTACCA | TTTC | chr18:+2424130 | | TATGACAACTT | CGAATCTATCCAG |
| | CTGCA (SEQ ID NO: 44) | | | | TCCCCTGGTC | GTGCTCAGT (SEQ |
| | | | | | A (SEQ ID | ID NO: 90) |
| | | | | | NO: 89) | |
| OT2 | CCATCAACCACAGCCA | TTTG | chr2:−213730258 | | ACCATCAGAG | GCTGCTCTTATGG |
| | CTGCA (SEQ ID NO: 45) | | | | GAATCCCATTT | TAAAAATATGCT |
| | | | | | C (SEQ ID | (SEQ ID NO: 92) |
| | | | | | NO: 91) | |
| OT3 | CCTTAATACACAGCCAT | TTTG | chr4:−78706036 | | GGTCTGGGTT | TTAAAGGCTTTGAG |
| | TGTG (SEQ ID NO: 46) | | | | TCATTCAAAAT | GCCAGTCC (SEQ |
| | | | | | GTG (SEQ ID | ID NO: 94) |
| | | | | | NO: 93) | |
| OT4 | CCATTTTACACAGTCAC | TTTA | chr6:−141264027 | | CCACTTGAGA | ATGTTGGATGGGG |
| | TGGC (SEQ ID NO: 47) | | | | AACATTCCGAT | TAGCAGG (SEQ ID |
| | | | | | GA (SEQ ID | NO: 96) |
| | | | | | NO: 95) | |
| OT5 | CCCTGATCCACAGCCC | TTTC | chr3:+134726770 | | CAAGTACTGG | CTCCGGCCGGCAG |
| | CAGCC (SEQ ID NO: 48) | | | | TCAATAAAGG | TT (SEQ ID |
| | | | | | GC (SEQ ID | NO: 98) |
| | | | | | NO: 97) | |
| OT6 | CCATGGAACACAGCCA | TTTC | chr12:−45062523 | | AGGTGTTTTAC | AGGCTATAAGGGA |
| | CATCT (SEQ ID NO: 49) | | | | AGATAAGGGT | AGATGGTCA (SEQ |
| | | | | | CA | ID NO: 100) |
| | | | | | (SEQ ID NO: 99) | |
| OT7 | CAAAGATACATAGCCT | TTTC | chr8:−18761247 | | ACGCAAATCAA | GTTTTAAGACTTTT |
| | CTGCT (SEQ ID NO: 50) | | | | ACGTCAGGT | TAGAGGCGAGT |
| | | | | | (SEQ ID | (SEQ ID NO: |
| | | | | | NO: 101) | 102) |
| OT8 | CCATTATAAACAGCCAT | TTTA | chr1:+6218614 | | CTGGCTGGGC | GGGAACAGTCCTC |
| | GGCT (SEQ ID NO: 51) | | | | CTCTTTGTAA | GGTCACT (SEQ ID |
| | | | | | (SEQ ID | NO: 104) |
| | | | | | NO: 103) | |
| OT9 | CCAGGATACGCTGCCA | TTTG | chr19:+39160005 | | ACTGGATCTGT | GCTGTTCAGCAAG |
| | CTTCC (SEQ ID NO: 52) | | | | GTCTTTATGGG | TAAGCTGG (SEQ |
| | | | | | C (SEQ ID | ID NO: 106) |
| | | | | | NO: 105) | |
| OT10 | CCCTGGAACACAGCCA | TTTA | chr17:−4721649 | | GATTCTGTTAG | ATCTGTGTGCAGG |
| | CAGCA (SEQ ID NO: 53) | | | | CTTGTGGGGG | CGAGTTAG (SEQ |
| | | | | | (SEQ ID | ID NO: 108) |
| | | | | | NO: 107) | |

| | | | | | Forward | Reverse |
|---|---|---|---|---|---|---|
| Cas9-3 | Binding sequence | PAM | Location | Gene | primer | primer |
| ON | ATACACAGCCACTGCG | TGG | chrX:−124346647 | SH2D1A | CTGGGAGTCA | CTCGCTGTCCCTC |
| | TCCA (SEQ ID NO: 34) | | | | GGTGGTTGAC | AGCAAAT (SEQ ID |
| | | | | | (SEQ ID | NO: 110) |
| | | | | | NO: 109) | |
| OT1 | GAACACAGCCACTGCG | CAG | chr17:−80925931 | | GTCTGGGGAC | CACTGGGACTGTG |
| | TCCA (SEQ ID NO: 54) | | | | TGTTAGACGC | CTGACAA (SEQ ID |
| | | | | | (SEQ ID | NO: 112) |
| | | | | | NO: 111) | |
| OT2 | ATTCACAGCAGCTGCG | TAG | chr18:+79278188 | | TTGCAGGTATA | CCATCGAGTTCGA |
| | TCCA (SEQ ID NO: 55) | | | | GCAAAAGTGG | GTGAAGC (SEQ ID |
| | | | | | CA (SEQ ID | NO: 114) |
| | | | | | NO: 113) | |
| OT3 | TGGCGCAGCCACTGCG | GGG | chr19:−1762160 | | GACCCCCATC | CAGCTTCTGTCCC |
| | TCCA (SEQ ID NO: 56) | | | | CGCCC | GACTCC |
| | | | | | (SEQ ID | (SEQ ID NO: |
| | | | | | NO: 115) | 116) |
| OT4 | CAACACAGCCACTGTG | GGG | chr17:+1156951 | | ATGGTGAGGC | GTGAGGCTGCGGT |
| | TCCA (SEQ ID NO: 57) | | | | TGGGATCTGA | GAGTTA (SEQ ID |
| | | | | | (SEQ ID | NO: 118) |
| | | | | | NO: 117) | |
| OT5 | ATACACACCTACTGTGT | AGG | chr1:+22127989 | | TGTTTGCGGT | CAACAGAGACCCA |
| | CCA (SEQ ID NO: 58) | | | | GGAGGACATT | GTGAGCA (SEQ ID |
| | | | | | (SEQ ID | NO: 120) |
| | | | | | NO:119) | |
| OT6 | TTGCACAGCCACTGCC | AGG | chr10:+78377254 | | GGTGCTCATT | GAAAGCAAGAGCT |
| | TCCA (SEQ ID NO: 59) | | | | GGCCACAGT | CCCCTCA (SEQ ID |
| | | | | | (SEQ ID | NO: 122) |
| | | | | | NO: 121) | |
| OT7 | AAAACCAGCCACCGCG | TAG | chr7:−98017877 | | TCAGGCTGAG | AAAAAGTCCCCCA |
| | TCCA (SEQ ID NO: 60) | | | | AAGGAACACG | AGGGCAG (SEQ ID |
| | | | | | (SEQ ID | NO: 124) |
| | | | | | NO: 123) | |
| OT8 | CTCCACAGCCGCTGCG | GGG | chr13:+36214593 | SOHLH2 | CAGTGCTCCT | CCCGGGTTCCGCG |
| | TCCC (SEQ ID NO: 61) | | | | GGCAGATAATT | TTGA (SEQ ID |
| | | | | | GAG (SEQ ID | NO: 126) |
| | | | | | NO: 125) | |
| — | CTCCCCAGCCACTGTG | TGG | chr12:−117110314 | | Not identified | Not identified |
| | TCCA (SEQ ID NO: 62) | | | | | |
| OT9 | CTTGACAGCCACTGTG | AGG | chr16:−13340167 | | CTGATTTAAGA | GGGAAAGACACCT |
| | TCCA (SEQ ID NO: 63) | | | | TGTGCTCAAC | GGCCAAATA (SEQ |
| | | | | | CTTC (SEQ ID | ID NO: 128) |
| | | | | | NO: 127) | |
| OT10 | AACCACAGCCAGTGCG | TGG | chr2:−25378890 | | GGCTTGGTCA | GCATTGGCAGACC |
| | TCCC (SEQ ID NO: 64) | | | | GCCCTGTAGT | CGACA |
| | | | | | C (SEQ ID | (SEQ ID NO: |
| | | | | | NO: 129) | 130) |

| | | Right | | | | | |
|---|---|---|---|---|---|---|---|
| | | half | Orien- | | | Forward | Reverse |
| TALEN | Left half site | site | tation | Location | Gene | primer | primer |
| ON | TGCCCAAGAGTCCACC | GGCT | L-13-R | chrX: | SH2D1A | CTGTTGTTGG | CTCACAGCACACA |
| | AGG | GTGTA | | 124346622 | | GGTGCTTCTCT | TAGGCAGTAC |
| | (SEQ ID NO: 65) | TCATG | | | | C | (SEQ ID NO: |
| | | GCAAA | | | | (SEQ ID | 132) |
| | | (SEQ | | | | NO: 131) | |
| | | ID | | | | | |
| | | NO: 66) | | | | | |
| OT1 | TTTGCCtTaATACACcaC | atCctTt | R-28-R | chr13: | | CCCTTTCTTCC | GAGACATTTGCTA |
| | C | TATCA | | 74015878 | | CAAGGTAGGT | GGGAGGATGAG |
| | (SEQ ID NO: 67) | TGGtA | | | | C (SEQ ID | (SEQ ID NO: |
| | | AA | | | | NO: 133) | 134) |
| | | (SEQ | | | | | |
| | | ID | | | | | |
| | | NO: 68) | | | | | |
| OT2 | TTgGCaATaATcCACAGg | tGCTG | R-11-R | chr10: | | AAAGGACCCT | CATAGTCTCCAAGT |
| | a | TGTAT | | 68622934 | | CCCGCCTTAG | ATCTCCCCAC |
| | (SEQ ID NO: 69) | CATGt | | | | (SEQ ID | (SEQ ID NO: |
| | | CAgA | | | | NO: 135) | 136) |
| | | (SEQ | | | | | |
| | | ID | | | | | |
| | | NO: 70) | | | | | |
| OT3 | TTTGCCAaaAgACACAta | tGtTGT | R-14-R | chr5: | | GCACCTTGCA | CCTGACATCTAACA |
| | C | tTATC | | 56408412 | | GAAAGCATTTT | CTGTAGATTAGTTT |
| | (SEQ ID NO: 71) | ATaGt | | | | CCC (SEQ ID | TGCC (SEQ ID |
| | | AAA | | | | NO: 137) | NO: 138) |
| | | (SEQ | | | | | |
| | | ID | | | | | |
| | | NO: 72) | | | | | |
| OT4 | TTcaCCATtATACACAtC | GGCC | R-23-R | chr3: | | CCCTCCTTTAT | GCACCAGCACACC |
| | C | GgGcA | | 136206633 | | CTTCCTCCACC | CAGCTAA (SEQ ID |
| | (SEQ ID NO: 73) | CCATG | | | | (SEQ ID | NO: 140) |
| | | GCttA | | | | NO: 139) | |
| | | (SEQ | | | | | |
| | | ID | | | | | |
| | | NO: 74) | | | | | |
| OT5 | TTTtCCATGAgACACAcC | GtCaG | R-13-R | chr3: | | CTTCTCTTGTG | CAAAGGCAGTTCT |
| | C | TtTATC | | 86273842 | | TCTTGAACTAC | GCAGTCACATG |
| | (SEQ ID NO: 75) | ATtGC | | | | ATCAACAATAG | (SEQ ID NO: |
| | | cAt | | | | G (SEQ ID | 142) |
| | | (SEQ | | | | NO: 141) | |
| | | ID | | | | | |
| | | NO: 76) | | | | | |
| OT6 | TTTtCCtTtATACACAa | GcaTa | R-17-R | chr16: | | GTGTGTGGTG | GTTCTCAAGCACT |
| | CC | TaTAT | | 87976124 | | TCATGGAACCT | GTCCGTGG (SEQ |
| | (SEQ ID NO: 77) | CtTGG | | | | TAC | ID NO: 144) |
| | | CAAA | | | | (SEQ ID | |
| | | (SEQ | | | | NO: 143) | |
| | | ID | | | | | |
| | | NO: 78) | | | | | |
| OT7 | TGCCCAgGAcagaACCtG | CCTG | L-20-L | chr1: | | ACACATGTTCT | GGGGCACAGACAT |
| | G | GTGGc | | 23792545 | | GGGCCACTAG | GGTGTTAC (SEQ |
| | (SEQ ID NO: 79) | CTCTT | | | | G | ID NO: 146) |
| | | GGGC | | | | (SEQ ID | |
| | | C | | | | NO: 145) | |
| | | (SEQ | | | | | |
| | | ID | | | | | |
| | | NO: 80) | | | | | |
| OT8 | TTTGCCATGAagCAaAG | GGCT | R-15-R | chr22: | | GGTGTCGTAG | TAGGAGGTTCTTG |
| | CC | GTGaA | | 25845903 | | CATTGCTGTAG | GGAAAGCCTTG |
| | (SEQ ID NO: 81) | TgAaG | | | | G | (SEQ ID NO: |
| | | GggAg | | | | (SEQ ID | 148) |
| | | (SEQ | | | | NO: 147) | |
| | | ID | | | | | |
| | | NO: 82) | | | | | |
| OT9 | TcaGCCATGgTgCcCAG | CCTG | R-19-L | chr18: | | CGCCACCACA | GAGTGAGGAAAAC |
| | Ct | GTGtA | | 12832398 | | CCTGGCTAAA | GCCACTCTCAA |
| | (SEQ ID NO: 83) | CTCcTt | | | | (SEQ ID | (SEQ ID NO: |
| | | GGtA | | | | NO: 149) | 150) |
| | | (SEQ | | | | | |
| | | ID | | | | | |
| | | NO: 84) | | | | | |
| OT10 | aTTGCCtTtATtCAaAt | GGCTa | R-17-R | chr10: | | CTCAGCATTCT | GGTGGTGGATTTT |
| | CC | TGTAT | | 16900359 | | GCTTTTAGGCT | ATGCTACGTGG |
| | (SEQ ID NO: 85) | CtaGG | | | | CC | (SEQ ID NO: |
| | | CAAA | | | | (SEQ ID | 152) |
| | | (SEQ | | | | NO: 151) | |
| | | ID | | | | | |
| | | NO: 86) | | | | | |

1. A guide RNA comprising 17 to 24 nucleotides which are complementary to exon 1 of a human SH2 Domain Containing 1A (SH2D1A) gene or the complement thereof.

2. The guide RNA of claim 1, wherein the 17 nucleotides to 24 nucleotides are complementary to SEQ ID NO: 3 or SEQ ID NO: 4.

3. The guide RNA according to claim 1, comprising 20-21 nucleotides which are complementary to exon 1 of human SH2D1A or the complement thereof.

4. The guide RNA according to claim 1, wherein the guide RNA further comprises a scaffold sequence for a Cas nuclease.

5. The guide RNA according to claim 1, wherein the guide RNA is a single guide RNA.

6. A guide RNA according to claim 1, wherein the sequence comprises one of SEQ ID NO. 5-12.

7. A guide RNA according to claim 1, wherein the sequence comprises SEQ ID NO. 9.

8. The guide RNA according to claim 1, wherein the sequence comprises one of SEQ ID NO. 13-18.

9. A guide RNA according to claim 1, comprising SEQ ID NO.13.

10. A ribonuclear protein (RNP) complex comprising a Cas enzyme and the guide RNA according to claim 1.

11. The RNP complex of claim 10, wherein the Cas enzyme comprises a spCas9 enzyme and wherein the guide RNA comprises a sequence set forth in one of SEQ ID NO: 5-12.

12. The ribonuclear protein (RNP complex of claim 11, wherein the spCas9 enzyme is a mutant spCas9.

13. The RNP complex of claim 10, wherein the Cas enzyme comprises a Cas12a enzyme and wherein the guide RNA comprises a sequence set forth in one of SEQ ID NO: 13-18.

14. The RNP complex of claim 13, wherein the Cas12a enzyme is mutant asCas12a.

15. A construct encoding the guide RNA of claim 1.

16. A gene editing kit comprising:
the ribonuclear protein complex of claim 10, and a donor sequence or a construct encoding for a donor sequence.

17. The gene editing kit of claim 16, comprising an Adeno-associated virus (AAV) construct encoding for a donor sequence, wherein the construct is an AAV construct.

18. The gene editing kit of claim 17, wherein the AAV construct is of serotype AAV6.

19. The gene editing kit of claim 16, wherein the donor sequence encodes for a SLAM-associated protein (SAP) protein.

20. An in vitro method of forming a double strand break within exon 1 of the human SH2D1A gene, the method comprising: contacting a cell with the RNP complex of claim 10.

21. An in vitro method of editing the SH2D1A gene, the method comprising: contacting a cell with the gene editing kit of claim 16.

22. The in vitro method of editing the SH2D1A gene according to claim 21, wherein the contacting comprises electroporation.

23. The in vitro method of claim 20, wherein the cell is a T cell.

24. The in vitro method of claim 20, wherein the cell is a haematopoietic stem cell.

25. The in vitro method of claim 20, wherein the contacting does not comprise serum.

26. The guide RNA of claim 1, an RNP complex comprising a Cas enzyme and the guide RNA, or a gene editing kit comprising the RNP complex and a donor sequence or a construct encoding for a donor sequence, for use in therapy.

27. The guide RNA of claim 1, an RNP complex comprising a Cas enzyme and the guide RNA, or a gene editing kit comprising the RNP complex and a donor sequence or a construct encoding for a donor sequence, for use in the treatment of an SAP-mediated disease.

28. The gene editing kit of claim 16, for use in the treatment of X-linked lymphoproliferative disease.


SEQUENCE LISTING (152 sequences)

SEQ ID NO: 1 - DNA, 137 bp, Homo sapiens
  source 1..137: mol_type = genomic DNA
atggacgcagtggctgtgtatcatggcaaaatcagcagggaaaccggcgagaagctcctgcttgccactgggctggatggcagctatttgctgagggacagcgagagcgtgccaggcgtgtactgcctatgtgtgct

SEQ ID NO: 2 - DNA, 137 bp, Homo sapiens
  source 1..137: mol_type = genomic DNA
agcacacataggcagtacacgcctggcacgctctcgctgtccctcagcaaatagctgccatccagcccagtggcaagcaggagcttctcgccggtttccctgctgattttgccatgatacacagccactgcgtccat

SEQ ID NO: 3 - DNA, 26 bp, Homo sapiens
  source 1..26: mol_type = genomic DNA
atggacgcagtggctgtgtatcatgg

SEQ ID NO: 4 - DNA, 26 bp, Homo sapiens
  source 1..26: mol_type = genomic DNA
ccatgatacacagccactgcgtccat

SEQ ID NO: 5 - RNA, 20 nt, synthetic construct
  misc_feature 1..20: note = Cas9-1
  source 1..20: mol_type = other RNA
gtatcatggcaaaatcagca

SEQ ID NO: 6 - RNA, 17 nt, synthetic construct
  misc_feature 1..17: note = Cas9-1t
  source 1..17: mol_type = other RNA
tcatggcaaaatcagca

SEQ ID NO: 7 - RNA, 20 nt, synthetic construct
  misc_feature 1..20: note = Cas9-2
  source 1..20: mol_type = other RNA
acacataggcagtacacgcc

SEQ ID NO: 8 - RNA, 17 nt, synthetic construct
  misc_feature 1..17: note = Cas9-2t
  source 1..17: mol_type = other RNA
cataggcagtacacgcc

SEQ ID NO: 9 - RNA, 20 nt, synthetic construct
  misc_feature 1..20: note = Cas9-3
  source 1..20: mol_type = other RNA
atacacagccactgcgtcca

SEQ ID NO: 10 - RNA, 17 nt, synthetic construct
  misc_feature 1..17: note = Cas9-3t
  source 1..17: mol_type = other RNA
cacagccactgcgtcca

SEQ ID NO: 11 - RNA, 20 nt, synthetic construct
  misc_feature 1..20: note = Cas9-4
  source 1..20: mol_type = other RNA
gacgcagtggctgtgtatca

SEQ ID NO: 12 - RNA, 17 nt, synthetic construct
  misc_feature 1..17: note = Cas9-4t
  source 1..17: mol_type = other RNA
gcagtggctgtgtatca

SEQ ID NO: 13 - RNA, 21 nt, synthetic construct
  misc_feature 1..21: note = Cas12a-1
  source 1..21: mol_type = other RNA
ccatgatacacagccactgcg

SEQ ID NO: 14 - RNA, 17 nt, synthetic construct
  misc_feature 1..17: note = Cas12a-1t
  source 1..17: mol_type = other RNA
ccatgatacacagccac

SEQ ID NO: 15 - RNA, 21 nt, synthetic construct
  misc_feature 1..21: note = Cas12a-2
  source 1..21: mol_type = other RNA
cctgctgattttgccatgata

SEQ ID NO: 16 - RNA, 17 nt, synthetic construct
  misc_feature 1..17: note = Cas12a-2t
  source 1..17: mol_type = other RNA
cctgctgattttgccat

SEQ ID NO: 17 - RNA, 21 nt, synthetic construct
  misc_feature 1..21: note = Cas12a-3
  source 1..21: mol_type = other RNA
ctgagggacagcgagagcgtg

SEQ ID NO: 18 - RNA, 17 nt, synthetic construct
  misc_feature 1..17: note = Cas12a-3t
  source 1..17: mol_type = other RNA
ctgagggacagcgagag

SEQ ID NO: 19 - DNA, 387 bp, synthetic construct
  misc_feature 1..387: note = coSAP DNA
  source 1..387: mol_type = other DNA
atggatgccgtggccgtgtaccacggcaagatcagcagagagacaggcgagaagctgctgctggccaccggcctggacggcagctacctgctgagggacagcgagagcgtgcccggcgtgtactgcctgtgcgtgctgtatcacggctacatctacacctaccgggtgtcccagaccgagacaggctcttggagcgccgagacagcccctggcgtgcacaagcggtacttccggaagatcaagaacctgatcagcgccttccagaagcccgaccagggcatcgtgatccccctgcagtaccccgtggaaaagaagtccagcgccagaagcacccagggcaccaccggaatccgcgaggaccccgacgtgtgcctgaaggccccctga

SEQ ID NO: 20 - DNA, 720 bp, synthetic construct
  misc_feature 1..720: note = GFP
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 21 - DNA, 850 bp, synthetic construct
  misc_feature 1..850: note = HAL
  source 1..850: mol_type = other DNA
aggcaaagcaccagttaaatgattcacagtctcatctaaagcccatcaaatagatacatctggaacatctttggatgtacattgtgacttttgtatctcaactagcactttgggaccttggacaaggtgttaggaaagaaatcaatatttgctcaggatcaagacttctcaagatgactgcgtgaggtagaaggaagagggagaagaagaacttgcccagagttcactgttcatctactatgtctgattctgtaggagtcttcatggccatgttcagtaatgattgcatactgacagtactgataaaactatcagtgttttgttctcagagctcttggtcctttcattttgtcagtgtgtttttgccctacgtagtgggtccacataccaacagagacatgtacaacttgcctctatcatcaactccttatgggctagttgagctaagttattcctggtggcctctgagtaaaccgcaagaacagaagcattactaagcatccccgtctgagtgcaagggtgtgtgttggcagtacagccccaatcttgcaaaatccttcttccaatgttcctcccctctctgtatgaaccctgtgttggggggcagaagatggaagcccttggcaagctcgatcgaaccaagctactaaattgctgagctcgttttaactgaagtgtgagaaggaggtttaaggcaagtagacaacatcctgttgttggggtgcttctctcttttttgcacatctggctgaactgggagtcaggtggttgacttgtgcctggctgcagtagcagcggcatctcccttgcacagttctcctcctcggcctgcccaagagtccaccaggcc

SEQ ID NO: 22 - DNA, 850 bp, synthetic construct
  misc_feature 1..850: note = HAR
  source 1..850: mol_type = other DNA
tggacgcagtggctgtgtatcatggcaaaatcagcagggaaaccggcgagaagctcctgcttgccactgggctggatggcagctatttgctgagggacagcgagagcgtgccaggcgtgtactgcctatgtgtgctgtgagtatgatacggtggacatgggcctgctgagggtgtgggcggtgggcaacagcagctggggccagggtggaggccgaggcaggcaggggcgccggcgttagcagctcgccgacgcctcctccgtggcccaccctcaagtccagcccagggccgtggtggaacacactttcgcctcaatccctcgctggggatttcattccctgggcctcctgctctccagaaggccctcaccagagccttttggggcagctgcagagaatgccatttcgggtggtgaaatgtggtccacagtttaccttcgctccgggaaagacccaggctccccacttccattttgacccctatcgggtggcgccctgcgagccttactctagctctctttaaacgacttttgaagtgtcatcccaaaggaagagcatgacattcagagagaggaggcacattaagaaacagagtcaattactttaagaatgcaatgccagttaggtcgagatgctgagagaggagggggatgtgtgtcagtaaaatcacctctctggatctgctgatgtagcatcctttagaaaagtagtggggggtggggaggggcaggggatttcagaaataggacccttcttgataaacgttaggactcacaatagtcttaaagggaatggagggaaaacacccttgttatgattcagctttgccatttgcctaggcatgtctcat

SEQ ID NO: 23 - DNA, 134 bp, synthetic construct
  misc_feature 1..134: note = SV40pA
  source 1..134: mol_type = other DNA
ttgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 24 - DNA, 227 bp, synthetic construct
  misc_feature 1..227: note = bGHpA
  source 1..227: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggt

SEQ ID NO: 25 - DNA, 1764 bp, synthetic construct
  misc_feature 1..1764: note = SAP UTR
  source 1..1764: mol_type = other DNA
agaaaaataaaacaccttgtactttattttctataatttaaatatatgctaagtcttatatattgtagataatacagttcggtgagctacaaatgcatttctaaagccattgtagtcctgtaatggaagcatctagcatgtcgtcaaagctgaaatggacttttgtacatagtgaggagctttgaaacgaggattgggaaaaagtaattccgtaggttattttcagttattatatttacaaatgggaaacaaaaggataatgaatactttataaaggattaatgtcaattcttgccaaatataaataaaaataatcctcagtttttgtgaaaagctccatttttagtgaaatattattttatagctactaattttaaaatgtcttgcttgattgtatggtgggaagttggctggtgtcccttgtctttgccaagttctccactagctatggtgtcataggctcttttgggatttttgaagctgtatactgtgtgctaaaacaagcactaaacaaagagtgaaggatttatgtttaattctgaaagcaaccttcttgcctagtgttctgatattggacagtaaaatccacagaccaacctggagttgaaaatcttataatttaaaatatgctctaaacatgtttatcgtatttgatgctacaggatttgaaattgtattacaaatccaatgaaatgagtttttcttttcatttacctctgccccagttgtttctactacatggaagacctcattttgaagggaaatttcagcagctgcagctcatgagtaactgatttgtaacaagcctccttttaaagtaaccctacaaaaccactggaaagtttatggttgtattattttttaaaaaaattccaagtgattgaaacctacacgagatacagaattttatgcggcattttcttctcacatttatatttttgtgattttgtgattgattatatgtcactttgctacagggctcacagaattcattcactcaacaaacataatagggcgctgagggcatagaagtaaaaacacctggtccctgctctcagttcactgtcttgttggacgagaaaacaataacgataaaagacagtgaaagaaaataacgataaaagacagtgaaagaaaataacaataaaagacaaggaaaaaataacaatgaaagttgataagtacatgataagcgaggttccccgtgtgtaggtagatctggtctttagaggcagatagataggtcagtgcaaatactctggtccatgggccatatgaaaaggctaagcttcactgtaaaataataactgggaattctggattgtgtatgggtgttggtgaacttggttttaattagtgaactgctgagagacagagctattctccatgtactggcaagacctgatttctgagcatttaatatggatgccgtgggagtacaaaagtggagtgtggcctgagtaatgcattatgggtggtttaccatttcttgaggtaaaagcatcacatgaacttgtaaaggaatttaaaaatcctactttcataataagttgcataggtttaataatttttaattatatggcttgagtttaaattgtaataggcgtaactaattttaactctataatgtgttcattctggaataatcctaaacatatgaattatgtttgcatgttcacttccaagagcctttttttgaaaaaaagctttttttgaatcatcaagtctttcacatttaaataaagtgtttgaaagctttattta

SEQ ID NO: 26 - DNA, 592 bp, synthetic construct
  misc_feature 1..592: note = WPREmut6
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 27 - DNA, 3655 bp, synthetic construct
  misc_feature 1..3655: note = G7bc
  source 1..3655: mol_type = other DNA
aggcaaagcaccagttaaatgattcacagtctcatctaaagcccatcaaatagatacatctggaacatctttggatgtacattgtgacttttgtatctcaactagcactttgggaccttggacaaggtgttaggaaagaaatcaatatttgctcaggatcaagacttctcaagatgactgcgtgaggtagaaggaagagggagaagaagaacttgcccagagttcactgttcatctactatgtctgattctgtaggagtcttcatggccatgttcagtaatgattgcatactgacagtactgataaaactatcagtgttttgttctcagagctcttggtcctttcattttgtcagtgtgtttttgccctacgtagtgggtccacataccaacagagacatgtacaacttgcctctatcatcaactccttatgggctagttgagctaagttattcctggtggcctctgagtaaaccgcaagaacagaagcattactaagcatccccgtctgagtgcaagggtgtgtgttggcagtacagccccaatcttgcaaaatccttcttccaatgttcctcccctctctgtatgaaccctgtgttggggggcagaagatggaagcccttggcaagctcgatcgaaccaagctactaaattgctgagctcgttttaactgaagtgtgagaaggaggtttaaggcaagtagacaacatcctgttgttggggtgcttctctcttttttgcacatctggctgaactgggagtcaggtggttgacttgtgcctggctgcagtagcagcggcatctcccttgcacagttctcctcctcggcctgcccaagagtccaccaggcctgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacgcgtctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcggccgcactagttgctttatttgtgaaatttgtgatgctattgctttatttgtaaccattataagctgcaataaacaagttaacaacaacaattgcattcattttatgtttcaggttcagggggaggtgtgggaggttttttaaatggacgcagtggctgtgtatcatggcaaaatcagcagggaaaccggcgagaagctcctgcttgccactgggctggatggcagctatttgctgagggacagcgagagcgtgccaggcgtgtactgcctatgtgtgctgtgagtatgatacggtggacatgggcctgctgagggtgtgggcggtgggcaacagcagctggggccagggtggaggccgaggcaggcaggggcgccggcgttagcagctcgccgacgcctcctccgtggcccaccctcaagtccagcccagggccgtggtggaacacactttcgcctcaatccctcgctggggatttcattccctgggcctcctgctctccagaaggccctcaccagagccttttggggcagctgcagagaatgccatttcgggtggtgaaatgtggtccacagtttaccttcgctccgggaaagacccaggctccccacttccattttgacccctatcgggtggcgccctgcgagccttactctagctctctttaaacgacttttgaagtgtcatcccaaaggaagagcatgacattcagagagaggaggcacattaagaaacagagtcaattactttaagaatgcaatgccagttaggtcgagatgctgagagaggagggggatgtgtgtcagtaaaatcacctctctggatctgctgatgtagcatcctttagaaaagtagtggggggtggggaggggcaggggatttcagaaataggacccttcttgataaacgttaggactcacaatagtcttaaagggaatggagggaaaacacccttgttatgattcagctttgccatttgcctaggcatgtctcat

SEQ ID NO: 28 - DNA, 3606 bp, synthetic construct
  misc_feature 1..3606: note = G15
  source 1..3606: mol_type = other DNA
aggcaaagcaccagttaaatgattcacagtctcatctaaagcccatcaaatagatacatctggaacatctttggatgtacattgtgacttttgtatctcaactagcactttgggaccttggacaaggtgttaggaaagaaatcaatatttgctcaggatcaagacttctcaagatgactgcgtgaggtagaaggaagagggagaagaagaacttgcccagagttcactgttcatctactatgtctgattctgtaggagtcttcatggccatgttcagtaatgattgcatactgacagtactgataaaactatcagtgttttgttctcagagctcttggtcctttcattttgtcagtgtgtttttgccctacgtagtgggtccacataccaacagagacatgtacaacttgcctctatcatcaactccttatgggctagttgagctaagttattcctggtggcctctgagtaaaccgcaagaacagaagcattactaagcatccccgtctgagtgcaagggtgtgtgttggcagtacagccccaatcttgcaaaatccttcttccaatgttcctcccctctctgtatgaaccctgtgttggggggcagaagatggaagcccttggcaagctcgatcgaaccaagctactaaattgctgagctcgttttaactgaagtgtgagaaggaggtttaaggcaagtagacaacatcctgttgttggggtgcttctctcttttttgcacatctggctgaactgggagtcaggtggttgacttgtgcctggctgcagtagcagcggcatctcccttgcacagttctcctcctcggcctgcccaagagtccaccaggccatggatgccgtggccgtgtaccacggcaagatcagcagagagacaggcgagaagctgctgctggccaccggcctggacggcagctacctgctgagggacagcgagagcgtgcccggcgtgtactgcctgtgcgtgctgtatcacggctacatctacacctaccgggtgtcccagaccgagacaggctcttggagcgccgagacagcccctggcgtgcacaagcggtacttccggaagatcaagaacctgatcagcgccttccagaagcccgaccagggcatcgtgatccccctgcagtaccccgtggaaaagaagtccagcgccagaagcacccagggcaccaccggaatccgcgaggaccccgacgtgtgcctgaaggcccccgctaccaacttttccctgctgaaacaggcaggcgatgtggaggaaaatcctgggccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcggccgcactagttgctttatttgtgaaatttgtgatgctattgctttatttgtaaccattataagctgcaataaacaagttaacaacaacaattgcattcattttatgtttcaggttcagggggaggtgtgggaggttttttaaatggacgcagtggctgtgtatcatggcaaaatcagcagggaaaccggcgagaagctcctgcttgccactgggctggatggcagctatttgctgagggacagcgagagcgtgccaggcgtgtactgcctatgtgtgctgtgagtatgatacggtggacatgggcctgctgagggtgtgggcggtgggcaacagcagctggggccagggtggaggccgaggcaggcaggggcgccggcgttagcagctcgccgacgcctcctccgtggcccaccctcaagtccagcccagggccgtggtggaacacactttcgcctcaatccctcgctggggatttcattccctgggcctcctgctctccagaaggccctcaccagagccttttggggcagctgcagagaatgccatttcgggtggtgaaatgtggtccacagtttaccttcgctccgggaaagacccaggctccccacttccattttgacccctatcgggtggcgccctgcgagccttactctagctctctttaaacgacttttgaagtgtcatcccaaaggaagagcatgacattcagagagaggaggcacattaagaaacagagtcaattactttaagaatgcaatgccagttaggtcgagatgctgagagaggagggggatgtgtgtcagtaaaatcacctctctggatctgctgatgtagcatcctttagaaaagtagtggggggtggggaggggcaggggatttcagaaataggacccttcttgataaacgttaggactcacaatagtcttaaagggaatggagggaaaacacccttgttatgattcagctttgccatttgcctaggcatgtctcat

SEQ ID NO: 29 - DNA, 4625 bp, synthetic construct
  misc_feature 1..4625: note = G16
  source 1..4625: mol_type = other DNA
aggcaaagcaccagttaaatgattcacagtctcatctaaagcccatcaaatagatacatctggaacatctttggatgtacattgtgacttttgtatctcaactagcactttgggaccttggacaaggtgttaggaaagaaatcaatatttgctcaggatcaagacttctcaagatgactgcgtgaggtagaaggaagagggagaagaagaacttgcccagagttcactgttcatctactatgtctgattctgtaggagtcttcatggccatgttcagtaatgattgcatactgacagtactgataaaactatcagtgttttgttctcagagctcttggtcctttcattttgtcagtgtgtttttgccctacgtagtgggtccacataccaacagagacatgtacaacttgcctctatcatcaactccttatgggctagttgagctaagttattcctggtggcctctgagtaaaccgcaagaacagaagcattactaagcatccccgtctgagtgcaagggtgtgtgttggcagtacagccccaatcttgcaaaatccttcttccaatgttcctcccctctctgtatgaaccctgtgttggggggcagaagatggaagcccttggcaagctcgatcgaaccaagctactaaattgctgagctcgttttaactgaagtgtgagaaggaggtttaaggcaagtagacaacatcctgttgttggggtgcttctctcttttttgcacatctggctgaactgggagtcaggtggttgacttgtgcctggctgcagtagcagcggcatctcccttgcacagttctcctcctcggcctgcccaagagtccaccaggccatggatgccgtggccgtgtaccacggcaagatcagcagagagacaggcgagaagctgctgctggccaccggcctggacggcagctacctgctgagggacagcgagagcgtgcccggcgtgtactgcctgtgcgtgctgtatcacggctacatctacacctaccgggtgtcccagaccgagacaggctcttggagcgccgagacagcccctggcgtgcacaagcggtacttccggaagatcaagaacctgatcagcgccttccagaagcccgaccagggcatcgtgatccccctgcagtaccccgtggaaaagaagtccagcgccagaagcacccagggcaccaccggaatccgcgaggaccccgacgtgtgcctgaaggcccccgctaccaacttttccctgctgaaacaggcaggcgatgtggaggaaaatcctgggccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagaaaaataaaacaccttgtactttattttctataatttaaatatatgctaagtcttatatattgtagataatacagttcggtgagctacaaatgcatttctaaagccattgtagtcctgtaatggaagcatctagcatgtcgtcaaagctgaaatggacttttgtacatagtgaggagctttgaaacgaggattgggaaaaagtaattccgtaggttattttcagttattatatttacaaatgggaaacaaaaggataatgaatactttataaaggattaatgtcaattcttgccaaatataaataaaaataatcctcagtttttgtgaaaagctccatttttagtgaaatattattttatagctactaattttaaaatgtcttgcttgattgtatggtgggaagttggctggtgtcccttgtctttgccaagttctccactagctatggtgtcataggctcttttgggatttttgaagctgtatactgtgtgctaaaacaagcactaaacaaagagtgaaggatttatgtttaattctgaaagcaaccttcttgcctagtgttctgatattggacagtaaaatccacagaccaacctggagttgaaaatcttataatttaaaatatgctctaaacatgtttatcgtatttgatgctacaggatttgaaattgtattacaaatccaatgaaatgagtttttcttttcatttacctctgccccagttgtttctactacatggaagacctcattttgaagggaaatttcagcagctgcagctcatgagtaactgatttgtaacaagcctccttttaaagtaaccctacaaaaccactggaaagtttatggttgtattattttttaaaaaaattccaagtgattgaaacctacacgagatacagaattttatgcggcattttcttctcacatttatatttttgtgattttgtgattgattatatgtcactttgctacagggctcacagaattcattcactcaacaaacataatagggcgctgagggcatagaagtaaaaacacctggtccctgctctcagttcactgtcttgttggacgagaaaacaataacgataaaagacagtgaaagaaaataacgataaaagacagtgaaagaaaataacaataaaagacaaggaaaaaataacaatgaaagttgataagtacatgataagcgaggttccccgtgtgtaggtagatctggtctttagaggcagatagataggtcagtgcaaatactctggtccatgggccatatgaaaaggctaagcttcactgtaaaataataactgggaattctggattgtgtatgggtgttggtgaacttggttttaattagtgaactgctgagagacagagctattctccatgtactggcaagacctgatttctgagcatttaatatggatgccgtgggagtacaaaagtggagtgtggcctgagtaatgcattatgggtggtttaccatttcttgaggtaaaagcatcacatgaacttgtaaaggaatttaaaaatcctactttcataataagttgcataggtttaataatttttaattatatggcttgagtttaaattgtaataggcgtaactaattttaactctataatgtgttcattctggaataatcctaaacatatgaattatgtttgcatgttcacttccaagagcctttttttgaaaaaaagctttttttgaatcatcaagtctttcacatttaaataaagtgtttgaaagctttatttatggacgcagtggctgtgtatcatggcaaaatcagcagggaaaccggcgagaagctcctgcttgccactgggctggatggcagctatttgctgagggacagcgagagcgtgccaggcgtgtactgcctatgtgtgctgtgagtatgatacggtggacatgggcctgctgagggtgtgggcggtgggcaacagcagctggggccagggtggaggccgaggcaggcaggggcgccggcgttagcagctcgccgacgcctcctccgtggcccaccctcaagtccagcccagggccgtggtggaacacactttcgcctcaatccctcgctggggatttcattccctgggcctcctgctctccagaaggccctcaccagagccttttggggcagctgcagagaatgccatttcgggtggtgaaatgtggtccacagtttaccttcgctccgggaaagacccaggctccccacttccattttgacccctatcgggtggcgccctgcgagccttactctagctctctttaaacgacttttgaagtgtcatcccaaaggaagagcatgacattcagagagaggaggcacattaagaaacagagtcaattactttaagaatgcaatgccagttaggtcgagatgctgagagaggagggggatgtgtgtcagtaaaatcacctctctggatctgctgatgtagcatcctttagaaaagtagtggggggtggggaggggcaggggatttcagaaataggacccttcttgataaacgttaggactcacaatagtcttaaagggaatggagggaaaacacccttgttatgattcagctttgccatttgcctaggcatgtctcat

SEQ ID NO: 30 - DNA, 20 bp, Homo sapiens
  source 1..20: mol_type = genomic DNA
gtatcatggcaaaatcagca

SEQ ID NO: 31 - DNA, 17 bp, Homo sapiens
  source 1..17: mol_type = genomic DNA
tcatggcaaaatcagca

SEQ ID NO: 32 - DNA, 20 bp, Homo sapiens
  source 1..20: mol_type = genomic DNA
acacataggcagtacacgcc

SEQ ID NO: 33 - DNA, 17 bp, Homo sapiens
  source 1..17: mol_type = genomic DNA
cataggcagtacacgcc

SEQ ID NO: 34 - DNA, 20 bp, Homo sapiens
  source 1..20: mol_type = genomic DNA
atacacagccactgcgtcca

SEQ ID NO: 35 - DNA, 17 bp, Homo sapiens
  source 1..17: mol_type = genomic DNA
cacagccactgcgtcca

SEQ ID NO: 36 - DNA, 20 bp, Homo sapiens
  source 1..20: mol_type = genomic DNA
gacgcagtggctgtgtatca

SEQ ID NO: 37 - DNA, 17 bp, Homo sapiens
  source 1..17: mol_type = genomic DNA
gcagtggctgtgtatca

SEQ ID NO: 38 - DNA, 21 bp, Homo sapiens
  source 1..21: mol_type = genomic DNA
ccatgatacacagccactgcg

SEQ ID NO: 39 - DNA, 17 bp, Homo sapiens
  source 1..17: mol_type = genomic DNA
ccatgatacacagccac

SEQ ID NO: 40 - DNA, 21 bp, Homo sapiens
  source 1..21: mol_type = genomic DNA
cctgctgattttgccatgata

SEQ ID NO: 41 - DNA, 17 bp, Homo sapiens
  source 1..17: mol_type = genomic DNA
cctgctgattttgccat

SEQ ID NO: 42 - DNA, 21 bp, Homo sapiens
  source 1..21: mol_type = genomic DNA
ctgagggacagcgagagcgtg

SEQ ID NO: 43 - DNA, 17 bp, Homo sapiens
  source 1..17: mol_type = genomic DNA
ctgagggacagcgagag

SEQ ID NO: 44 - DNA, 21 bp, Homo sapiens
  source 1..21: mol_type = genomic DNA
ccatgaaacactaccactgca

SEQ ID NO: 45 - DNA, 21 bp, Homo sapiens
  source 1..21: mol_type = genomic DNA
ccatcaaccacagccactgca

SEQ ID NO: 46 - DNA, 21 bp, Homo sapiens
  source 1..21: mol_type = genomic DNA
ccttaatacacagccattgtg

SEQ ID NO: 47 - DNA, 21 bp, Homo sapiens
  source 1..21: mol_type = genomic DNA
ccattttacacagtcactggc

SEQ ID NO: 48 - DNA, 21 bp, Homo sapiens
  source 1..21: mol_type = genomic DNA
ccctgatccacagccccagcc

SEQ ID NO: 49 - DNA, 21 bp, Homo sapiens
  source 1..21: mol_type = genomic DNA
ccatggaacacagccacatct

SEQ ID NO: 50 - DNA, 21 bp, Homo sapiens
  source 1..21: mol_type = genomic DNA
caaagatacatagcctctgct

SEQ ID NO: 51 - DNA, 21 bp, Homo sapiens
  source 1..21: mol_type = genomic DNA
ccattataaacagccatggct

SEQ ID NO: 52 - DNA, 21 bp, Homo sapiens
  source 1..21: mol_type = genomic DNA
ccaggatacgctgccacttcc

SEQ ID NO: 53 - DNA, 21 bp, Homo sapiens
  source 1..21: mol_type = genomic DNA
ccctggaacacagccacagca

SEQ ID NO: 54 - DNA, 20 bp, Homo sapiens
  source 1..20: mol_type = genomic DNA
gaacacagccactgcgtcca

SEQ ID NO: 55 - DNA, 20 bp, Homo sapiens
  source 1..20: mol_type = genomic DNA
attcacagcagctgcgtcca

SEQ ID NO: 56 - DNA, 20 bp, Homo sapiens
  source 1..20: mol_type = genomic DNA
tggcgcagccactgcgtcca

SEQ ID NO: 57 - DNA, 20 bp, Homo sapiens
  source 1..20: mol_type = genomic DNA
caacacagccactgtgtcca

SEQ ID NO: 58 - DNA, 20 bp, Homo sapiens
  source 1..20: mol_type = genomic DNA
atacacacctactgtgtcca

SEQ ID NO: 59 - DNA, 20 bp, Homo sapiens
  source 1..20: mol_type = genomic DNA
ttgcacagccactgcctcca

SEQ ID NO: 60 - DNA, 20 bp, Homo sapiens
  source 1..20: mol_type = genomic DNA
aaaaccagccaccgcgtcca

SEQ ID NO: 61 - DNA, 20 bp, Homo sapiens
  source 1..20: mol_type = genomic DNA
ctccacagccgctgcgtccc

SEQ ID NO: 62 - DNA, 20 bp, Homo sapiens
  source 1..20: mol_type = genomic DNA
ctccccagccactgtgtcca

SEQ ID NO: 63 - DNA, 20 bp, Homo sapiens
  source 1..20: mol_type = genomic DNA
cttgacagccactgtgtcca

SEQ ID NO: 64 - DNA, 20 bp, Homo sapiens
  source 1..20: mol_type = genomic DNA
aaccacagccagtgcgtccc

SEQ ID NO: 65 - DNA, 19 bp, Homo sapiens
  source 1..19: mol_type = genomic DNA
tgcccaagagtccaccagg

SEQ ID NO: 66 - DNA, 19 bp, Homo sapiens
  source 1..19: mol_type = genomic DNA
ggctgtgtatcatggcaaa

SEQ ID NO: 67 - DNA, 19 bp, Homo sapiens
  source 1..19: mol_type = genomic DNA
tttgccttaatacaccacc

SEQ ID NO: 68 - DNA, 19 bp, Homo sapiens
  source 1..19: mol_type = genomic DNA
atccttttatcatggtaaa

SEQ ID NO: 69 - DNA, 19 bp, Homo sapiens
  source 1..19: mol_type = genomic DNA
ttggcaataatccacagga

SEQ ID NO: 70 - DNA, 19 bp, Homo sapiens
  source 1..19: mol_type = genomic DNA
tgctgtgtatcatgtcaga

SEQ ID NO: 71 - DNA, 19 bp, Homo sapiens
  source 1..19: mol_type = genomic DNA
tttgccaaaagacacatac

SEQ ID NO: 72 - DNA, 19 bp, Homo sapiens
  source 1..19: mol_type = genomic DNA
tgttgtttatcatagtaaa

SEQ ID NO: 73 - DNA, 19 bp, Homo sapiens
  source 1..19: mol_type = genomic DNA
ttcaccattatacacatcc

SEQ ID NO: 74 - DNA, 19 bp, Homo sapiens
  source 1..19: mol_type = genomic DNA
ggccgggcaccatggctta

SEQ ID NO: 75 - DNA, 19 bp, Homo sapiens
  source 1..19: mol_type = genomic DNA
ttttccatgagacacaccc

SEQ ID NO: 76 - DNA, 19 bp, Homo sapiens
  source 1..19: mol_type = genomic DNA
gtcagtttatcattgccat

SEQ ID NO: 77 - DNA, 19 bp, Homo sapiens
  source 1..19: mol_type = genomic DNA
ttttcctttatacacaacc

SEQ ID NO: 78 - DNA, 19 bp, Homo sapiens
  source 1..19: mol_type = genomic DNA
gcatatatatcttggcaaa

SEQ ID NO: 79 - DNA, 19 bp, Homo sapiens
  source 1..19: mol_type = genomic DNA
tgcccaggacagaacctgg

SEQ ID NO: 80 - DNA, 19 bp, Homo sapiens
  source 1..19: mol_type = genomic DNA
cctggtggcctcttgggcc

SEQ ID NO: 81 - DNA, 19 bp, Homo sapiens
  source 1..19: mol_type = genomic DNA
tttgccatgaagcaaagcc

SEQ ID NO: 82 - DNA, 19 bp, Homo sapiens
  source 1..19: mol_type = genomic DNA
ggctgtgaatgaaggggag

SEQ ID NO: 83 - DNA, 19 bp, Homo sapiens
  source 1..19: mol_type = genomic DNA
tcagccatggtgcccagct

SEQ ID NO: 84 - DNA, 19 bp, Homo sapiens
  source 1..19: mol_type = genomic DNA
cctggtgtactccttggta

SEQ ID NO: 85 - DNA, 19 bp, Homo sapiens
  source 1..19: mol_type = genomic DNA
attgcctttattcaaatcc

SEQ ID NO: 86 - DNA, 19 bp, Homo sapiens
  source 1..19: mol_type = genomic DNA
ggctatgtatctaggcaaa

SEQ ID NO: 87 - DNA, 20 bp, synthetic construct
  misc_feature 1..20: note = Cas12a-1 ON Fwd primer
  source 1..20: mol_type = other DNA
ctgggagtcaggtggttgac

SEQ ID NO: 88 - DNA, 20 bp, synthetic construct
  misc_feature 1..20: note = Cas12a-1 ON Rev primer
  source 1..20: mol_type = other DNA
ctcgctgtccctcagcaaat

SEQ ID NO: 89 - DNA, 22 bp, synthetic construct
  misc_feature 1..22: note = Cas12a-1 OT1 Fwd primer
  source 1..22: mol_type = other DNA
tatgacaactttcccctggtca

SEQ ID NO: 90 - DNA, 22 bp, synthetic construct
  misc_feature 1..22: note = cas12a-1 OT1 Rev primer
  source 1..22: mol_type = other DNA
cgaatctatccaggtgctcagt

SEQ ID NO: 91 - DNA, 22 bp, synthetic construct
  misc_feature 1..22: note = Cas12a-2- OT2 Rev primer
  source 1..22: mol_type = other DNA
accatcagaggaatcccatttc

SEQ ID NO: 92 - DNA, 25 bp, synthetic construct
  misc_feature 1..25: note = Cas12a-2 OT2 Rev primer
  source 1..25: mol_type = other DNA
gctgctcttatggtaaaaatatgct

SEQ ID NO: 93 - DNA, 24 bp, synthetic construct
  misc_feature 1..24: note = Cas12a-1 OT3 Fwd primer
  source 1..24: mol_type = other DNA
ggtctgggtttcattcaaaatgtg

SEQ ID NO: 94 - DNA, 22 bp, synthetic construct
  misc_feature 1..22: note = Cas12a-1 OT3 Rev primer
  source 1..22: mol_type = other DNA
ttaaaggctttgaggccagtcc

SEQ ID NO: 95 - DNA, 23 bp, synthetic construct
  misc_feature 1..23: note = Cas12a-1 OT4 Fwd primer
  source 1..23: mol_type = other DNA
ccacttgagaaacattccgatga

SEQ ID NO: 96 - DNA, 20 bp, synthetic construct
  misc_feature 1..20: note = Cas12a-1 OT4 Rev primer
  source 1..20: mol_type = other DNA
atgttggatggggtagcagg

SEQ ID NO: 97 - DNA, 22 bp, synthetic construct
  misc_feature 1..22: note = Cas12a-1 OT5 Fwd primer
  source 1..22: mol_type = other DNA
caagtactggtcaataaagggc

SEQ ID NO: 98 - DNA, 15 bp, synthetic construct
  misc_feature 1..15: note = Cas12a-1 OT5 Rev primer
  source 1..15: mol_type = other DNA
ctccggccggcagtt

SEQ ID NO: 99 - DNA, 23 bp, synthetic construct
  misc_feature 1..23: note = Cas12a-1 OT6 Fwd primer
  source 1..23: mol_type = other DNA
aggtgttttacagataagggtca

SEQ ID NO: 100 - DNA, 22 bp, synthetic construct
  misc_feature 1..22: note = Cas12a-1 OT6 Rev primer
  source 1..22: mol_type = other DNA
aggctataagggaagatggtca

SEQ ID NO: 101 - DNA, 20 bp, synthetic construct
  misc_feature 1..20: note = Cas12a-1 OT7 Fwd primer
  source 1..20: mol_type = other DNA
acgcaaatcaaacgtcaggt

SEQ ID NO: 102 - DNA, 25 bp, synthetic construct
  misc_feature 1..25: note = Cas12a-1 OT7 Rev primer
  source 1..25: mol_type = other DNA
gttttaagactttttagaggcgagt

SEQ ID NO: 103 - DNA, 20 bp, synthetic construct
  misc_feature 1..20: note = Cas12a-1 OT8 Fwd primer
  source 1..20: mol_type = other DNA
ctggctgggcctctttgtaa

SEQ ID NO: 104 - DNA, 20 bp, synthetic construct
  misc_feature 1..20: note = Cas12a-1 OT8 Rev primer
  source 1..20: mol_type = other DNA
gggaacagtcctcggtcact

SEQ ID NO: 105 - DNA, 23 bp, synthetic construct
  misc_feature 1..23: note = Cas12a-1 OT9 Fwd primer
  source 1..23: mol_type = other DNA
actggatctgtgtctttatgggc

SEQ ID NO: 106 - DNA, 21 bp, synthetic construct
  misc_feature 1..21: note = Cas12a-1 OT9 Rev primer
  source 1..21: mol_type = other DNA
gctgttcagcaagtaagctgg

SEQ ID NO: 107 - DNA, 21 bp, synthetic construct
  misc_feature 1..21: note = Cas12a-1 OT10 Fwd primer
  source 1..21: mol_type = other DNA
gattctgttagcttgtggggg

SEQ ID NO: 108 - DNA, 21 bp, synthetic construct
  misc_feature 1..21: note = Cas12a-1 OT10 Rev primer
  source 1..21: mol_type = other DNA
atctgtgtgcaggcgagttag

SEQ ID NO: 109 - DNA, 20 bp, synthetic construct
  misc_feature 1..20: note = Cas9-3 ON Fwd primer
  source 1..20: mol_type = other DNA
ctgggagtcaggtggttgac

SEQ ID NO: 110 - DNA, 20 bp, synthetic construct
  misc_feature 1..20: note = Cas9-3 ON Rev primer
  source 1..20: mol_type = other DNA
ctcgctgtccctcagcaaat

SEQ ID NO: 111 - DNA, 20 bp, synthetic construct
  misc_feature 1..20: note = Cas9-3 OT1 Fwd primer
  source 1..20: mol_type = other DNA
gtctggggactgttagacgc

SEQ ID NO: 112 - DNA, 20 bp, synthetic construct
  misc_feature 1..20: note = Cas9-3 OT1 Rev primer
  source 1..20: mol_type = other DNA
cactgggactgtgctgacaa

SEQ ID NO: 113 - DNA, 23 bp, synthetic construct
  misc_feature 1..23: note = Cas9-3 OT2 Fwd primer
  source 1..23: mol_type = other DNA
ttgcaggtatagcaaaagtggca

SEQ ID NO: 114 - DNA, 20 bp, synthetic construct
  misc_feature 1..20: note = Cas9-3 OT2 Rev primer
  source 1..20: mol_type = other DNA
ccatcgagttcgagtgaagc

SEQ ID NO: 115 - DNA, 15 bp, synthetic construct
  misc_feature 1..15: note = Cas9-3 OT3 Fwd primer
  source 1..15: mol_type = other DNA
gacccccatccgccc

SEQ ID NO: 116 - DNA, 19 bp, synthetic construct
  misc_feature 1..19: note = Cas9-3 OT3 Rev primer
  source 1..19: mol_type = other DNA
cagcttctgtcccgactcc

SEQ ID NO: 117 - DNA, 20 bp, synthetic construct
  misc_feature 1..20: note = Cas9-3 OT4 Fwd primer
  source 1..20: mol_type = other DNA
atggtgaggctgggatctga

SEQ ID NO: 118 - DNA, 19 bp, synthetic construct
  misc_feature 1..19: note = Cas9-3 OT4 Rev primer
  source 1..19: mol_type = other DNA
gtgaggctgcggtgagtta

SEQ ID NO: 119 - DNA, 20 bp, synthetic construct
  misc_feature 1..20: note = Cas9-3 OT5 Fwd primer
  source 1..20: mol_type = other DNA
tgtttgcggtggaggacatt

SEQ ID NO: 120 - DNA, 20 bp, synthetic construct
  misc_feature 1..20: note = Cas9-3 OT5 Rev primer
  source 1..20: mol_type = other DNA
caacagagacccagtgagca

SEQ ID NO: 121 - DNA, 19 bp, synthetic construct
  misc_feature 1..19: note = Cas9-3 OT6 Fwd primer
  source 1..19: mol_type = other DNA
ggtgctcattggccacagt

SEQ ID NO: 122 - DNA, 20 bp, synthetic construct
  misc_feature 1..20: note = Cas9-3 OT6 Rev primer
  source 1..20: mol_type = other DNA
gaaagcaagagctcccctca

SEQ ID NO: 123 - DNA, 20 bp, synthetic construct
  misc_feature 1..20: note = Cas9-3 OT7 Fwd primer
  source 1..20: mol_type = other DNA
tcaggctgagaaggaacacg

SEQ ID NO: 124 - DNA, 20 bp, synthetic construct
  misc_feature 1..20: note = Cas9-3 OT7 Rev primer
  source 1..20: mol_type = other DNA
aaaaagtcccccaagggcag

SEQ ID NO: 125 - DNA, 24 bp, synthetic construct
  misc_feature 1..24: note = Cas9-3 OT8 Fwd primer
  source 1..24: mol_type = other DNA
cagtgctcctggcagataattgag

SEQ ID NO: 126 - DNA, 17 bp, synthetic construct
  misc_feature 1..17: note = Cas9-3 OT8 Rev primer
  source 1..17: mol_type = other DNA
cccgggttccgcgttga

SEQ ID NO: 127 - DNA, 25 bp, synthetic construct
  misc_feature 1..25: note = Cas9-3 OT9 Fwd primer
  source 1..25: mol_type = other DNA
ctgatttaagatgtgctcaaccttc

SEQ ID NO: 128 - DNA, 22 bp, synthetic construct
  misc_feature 1..22: note = Cas9-3 OT9 Rev primer
  source 1..22: mol_type = other DNA
gggaaagacacctggccaaata

SEQ ID NO: 129 - DNA, 21 bp, synthetic construct
  misc_feature 1..21: note = Cas9-10 OT10 Fwd primer
  source 1..21: mol_type = other DNA
ggcttggtcagccctgtagtc

SEQ ID NO: 130 - DNA, 18 bp, synthetic construct
  misc_feature 1..18: note = Cas9-10 OT10 Rev primer
  source 1..18: mol_type = other DNA
gcattggcagacccgaca

SEQ ID NO: 131 - DNA, 22 bp, synthetic construct
  misc_feature 1..22: note = TALEN ON Fwd primer
  source 1..22: mol_type = other DNA
ctgttgttggggtgcttctctc

SEQ ID NO: 132 - DNA, 23 bp, synthetic construct
  misc_feature 1..23: note = TALEN ON Rev primer
  source 1..23: mol_type = other DNA
ctcacagcacacataggcagtac

SEQ ID NO: 133 - DNA, 22 bp, synthetic construct
  misc_feature 1..22: note = TALEN OT1 Fwd primer
  source 1..22: mol_type = other DNA
ccctttcttcccaaggtaggtc

SEQ ID NO: 134 - DNA, 24 bp, synthetic construct
  misc_feature 1..24: note = TALEN OT1 Rev primer
  source 1..24: mol_type = other DNA
gagacatttgctagggaggatgag

SEQ ID NO: 135 - DNA, 20 bp, synthetic construct
  misc_feature 1..20: note = TALEN OT2 Fwd primer
  source 1..20: mol_type = other DNA
aaaggaccctcccgccttag

SEQ ID NO: 136 - DNA, 24 bp, synthetic construct
  misc_feature 1..24: note = TALEN OT2 Rev primer
  source 1..24: mol_type = other DNA
catagtctccaagtatctccccac

SEQ ID NO: 137 - DNA, 24 bp, synthetic construct
  misc_feature 1..24: note = TALEN OT3 Fwd primer
  source 1..24: mol_type = other DNA
gcaccttgcagaaagcattttccc

SEQ ID NO: 138 - DNA, 32 bp, synthetic construct
  misc_feature 1..32: note = TALEN OT3 Rev primer
  source 1..32: mol_type = other DNA
cctgacatctaacactgtagattagttttgcc

SEQ ID NO: 139 - DNA, 22 bp, synthetic construct
  misc_feature 1..22: note = TALEN OT4 Fwd primer
  source 1..22: mol_type = other DNA
ccctcctttatcttcctccacc

SEQ ID NO: 140 - DNA, 20 bp, synthetic construct
  misc_feature 1..20: note = TALEN OT4 Rev primer
  source 1..20: mol_type = other DNA
gcaccagcacacccagctaa

SEQ ID NO: 141 - DNA, 34 bp, synthetic construct
  misc_feature 1..34: note = TALEN OT5 Fwd primer
  source 1..34: mol_type = other DNA
cttctcttgtgtcttgaactacatcaacaatagg

SEQ ID NO: 142 - DNA, 24 bp, synthetic construct
  misc_feature 1..24: note = TALEN OT5 Rev primer
  source 1..24: mol_type = other DNA
caaaggcagttctgcagtcacatg

SEQ ID NO: 143 - DNA, 24 bp, synthetic construct
  misc_feature 1..24: note = TALEN OT6 Fwd primer
  source 1..24: mol_type = other DNA
gtgtgtggtgtcatggaaccttac

SEQ ID NO: 144 - DNA, 21 bp, synthetic construct
  misc_feature 1..21: note = TALEN OT6 Rev primer
  source 1..21: mol_type = other DNA
gttctcaagcactgtccgtgg

SEQ ID NO: 145 - DNA, 22 bp, synthetic construct
  misc_feature 1..22: note = TALEN OT7 Fwd primer
  source 1..22: mol_type = other DNA
acacatgttctgggccactagg

SEQ ID NO: 146 - DNA, 21 bp, synthetic construct
  misc_feature 1..21: note = TALEN OT7 Rev primer
  source 1..21: mol_type = other DNA
ggggcacagacatggtgttac

SEQ ID NO: 147 - DNA, 22 bp, synthetic construct
  misc_feature 1..22: note = TALEN OT8 Fwd primer
  source 1..22: mol_type = other DNA
ggtgtcgtagcattgctgtagg

SEQ ID NO: 148 - DNA, 24 bp, synthetic construct
  misc_feature 1..24: note = TALEN OT8 Rev primer
  source 1..24: mol_type = other DNA
taggaggttcttgggaaagccttg

SEQ ID NO: 149 - DNA, 20 bp, synthetic construct
  misc_feature 1..20: note = TALEN OT9 Fwd primer
  source 1..20: mol_type = other DNA
cgccaccacacctggctaaa

SEQ ID NO: 150 - DNA, 24 bp, synthetic construct
  misc_feature 1..24: note = TALEN OT9 Rev primer
  source 1..24: mol_type = other DNA
gagtgaggaaaacgccactctcaa

SEQ ID NO: 151 - DNA, 24 bp, synthetic construct
  misc_feature 1..24: note = TALEN OT10 Fwd primer
  source 1..24: mol_type = other DNA
ctcagcattctgcttttaggctcc

SEQ ID NO: 152 - DNA, 24 bp, synthetic construct
  misc_feature 1..24: note = TALEN OT10 Rev primer
  source 1..24: mol_type = other DNA
ggtggtggattttatgctacgtgg